(19) United States
(12) Patent Application Publication
(10) Pub. No.: US 2007/0148144 A1
(43) Pub. Date: Jun. 28, 2007

(54) Gene-enhanced tissue engineering

(76) Inventors: James M. Mason, Bethpage, NY (US); Paul C. Edwards, Elkhorn, NE (US); Daniel A. Grande, Sea Cliff, NJ (US)

Correspondence Address: AMSTER, ROTHSTEIN & EBENSTEIN LLP, 90 PARK AVENUE, NEW YORK, NY, 10016, US

(21) Appl. No.: 11/588,768

(22) Filed: Oct. 26, 2006

Related U.S. Application Data
(60) Provisional application No. 60/730,569, filed on Oct. 27, 2005.

(51) Int. Cl. A61K 48/00; A61K 31/522; C12N 5/08

(52) U.S. Cl. 424/93.21; 435/325; 435/366; 514/12; 514/263.31

(57) ABSTRACT

Provided are mammalian cells comprising a recombinant sonic hedgehog (SHH) gene such that a recombinant SHH protein can be expressed by the cell. Also provided are matrices suitable for applying to a tissue defect. Additionally provided are tissue regeneration compositions. Methods of regenerating tissue at the site of a tissue defect in a mammal are also provided.

### CROSS-REFERENCE TO RELATED APPLICATION

This application claims the benefit of U.S. Provisional Application No. 60/730,569, Filed Oct. 27, 2005.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. DE015430 awarded by The National Institutes of Health.

### BACKGROUND OF THE INVENTION

(1) Field of the Invention

The present invention generally relates to tissue engineering. More specifically, the invention provides compositions and methods for improved tissue engineering, using cells expressing a recombinant sonic hedgehog protein.

(2) Description of the Related Art References

Alzghoul M B, Gerrard D, Watkins B A, Hannon K. Ectopic expression of IGF-1 and SHH by skeletal muscle inhibit disuse-mediated skeletal muscle atrophy and bone osteopenia in vivo. FASEB J. 2004: 18(1); 221-223.

Anderson, D. G., Burdick, J. A., and Langer, R. Smart biomaterials. Science 305:1923-1924, 2004.

Arinzeh, T. L., Peter, S. J., Archambault, M. P., van den Bos, C., Gordon, S., Kraus, K., Smith, A., and Kadiyala, S. Allogeneic mesenchymal stem cells regenerate bone in a critical-sized canine segmental defect. J. Bone Joint Surg. Am. 2003: 85:1927-1935, 2003.

Awad H A, Butler D L, Harris M T, Ibrahim R E, Wu Y, Young R G, Kadiyala S, Boivin G P. In vitro characterization of mesenchymal stem cell-seeded collagen scaffolds for tendon repair: effects of initial seeding density on contraction kinetics. J Biomed Mater Res 2000: 51; 233-240.

Bederson, J. B., Pitts, L. H., Tsuji, M., Nishimura, M. C., Davis, R. L., Bartkowski, H. Rat middle cerebral artery occlusion: evaluation of the model and development of a neurologic examination. Stroke 17(3):472-476, 1986.

Bitgood M J & McMahon A P. Hedgehog and BMP genes are co-expressed at many diverse sites of cell-cell interaction in the mouse embryo. Dev Biol 1995; 172: 126-138.

Borgens, R. B. Mice regrow the tips of their foretoes. Science 217:747-750, 1982.

Breitbart A S, Grande D A, and Mason J M. Tissue Engineering and Gene Therapy in Facial Plastic Surgery. Cur. Opinion in Otolaryngology and Head and Neck Surg. 6:226-231, 1998.

Breitbart A S, Grande D A, Mason J M, James T, and Grant R T. Gene Modified Tissue Engineering: Gene Enhanced Tissue Engineering: Applications for Bone Healing Using Cultured Periosteal Cells Retrovirally Transduced with BMP-7 Gene. Ann Plast Surg. 42:488-495, 1999a.

Breitbart, A. S., Mason, J. M., Urmacher, C., Barcia, M., Grant, R. T., Pergolizzi, R. G., and Grande, D. A. Gene Enhanced Tissue Engineering: Applications for Wound Healing Using Cultured Dermal Fibroblasts Transduced Retrovirally with the PDGF-B Gene. Ann Plast Surg. 43:632-639, 1999b.

Breitbart, A S, Grande, D A, Laser, J, Barcia, M, Porti, D, Malhotra, S, Kogon, A, Grant, R T, and Mason, J M. Treatment of Ischemic Wounds Using Cultured Dermal Fibroblasts Transduced Retrovirally with PDGF-B and VegF121 Genes. Ann Plast Surg 46:555-562, 2001.

Breitbart, A. S., Laser, J, Parrett, B., Porti, D., Grant, R. T., Grande, D. A., and Mason, J. M. Accelerated Diabetic Wound Healing Using Cultured Dermal Fibroblasts Retrovirally Transduced with the PDGF-B Gene. Ann Plast Surg 51:409-414, 2003.

Brockes, J. P. Amphibian limb regeneration: rebuilding a complex structure. Science 276:81-87, 1997.

Bucala, R., Spiegel, L. A., Chesney, J., Hogan, M., and Cerami, A. Circulating fibrocytes define a new leukocyte subpopulation that mediates tissue repair. Mol Med 1:71-81, 1994.

Chang, H. Y., Sneddon, J. B., Alizadeh, A. A., Sood, R., West, R. B., Montgomery, K., Chi, J-T., ban de Rijn, M., Botstein, D., and Brown, P. O. Gene expression signature of fibroblast serum response predicts human cancer progression: similarities between tumors and wounds. PLoS Biol 2(2):e39, 2004.

Chen, Y. M., Chien, C. T., Hu-Tsai, M.I. et al. Pentoxifylline attenuates experimental mesangial proliferative glomerulonephritis. Kidney Int 56:932-943, 1999.

Chiang C et al. Cyclopia and defective axial patterning in mice lacking sonic hedgehog gene function. Nature 1996; 383: 407-413.

Cornell C N. Osteoconductive materials and their role as substitutes for autogenous bone grafts. Orthop Clin North Am 1999: 30(4); 591-598.

Craven, S. E., Lim, K. C., Ye, W., Engel, J. D., de Sauvage, F., and Rosenthal, A. Gata2 specifies serotonergic neurons downstream of sonic hedgehog. Development 131(5):1165-1173, 2004.

Dassule H R et al. Sonic hedgehog regulates growth and morphogenesis of the tooth. Development 2000; 127: 4775-4785.

Diduch D R, Jordan L C, Mierisch C M & Balian G. Marrow stromal cells embedded in alginate for repair of osteochondral defects. J Arthroscop Relat Surg 2000; 16: 571-577.

Dragoo J L, Choi J Y, Lieberman J R, Huang J, Zuk P A, Zhang J, Hedrick M H, Benhaim P. Bone induction by BMP-2 transduced stem cells derived from human fat. J Orthop Res. 2003: 21; 622-629.

Duncan, M. R., Hasan, A., Berman, B. Pentoxifylline, pentifylline, and interferons decrease type I and III procollagen mRNA levels in dermal fibroblasts: evidence for mediation by nuclear factor 1 down-regulation. J Invest Dermatol 104:282-286, 1995.

Dvorak, H. F. Tumors: Wounds that do not heal: Similarities between tumor stroma generation and wound healing. N Engl J Med 315:1650-1659, 1986.

Ebara S & Nakayama K. Mechanisms for the action of bone morphogenetic proteins and regulation of their activity. Spine 2002; 27: S10-S15.

Edwards, P., Ruggiero, S., Fantasia, J., Burakoff, R., Moorji, S., Razzano, P., Grande, D. A., and Mason, J. M. Sonic Hedgehog Gene Enhanced Tissue Engineering for Bone Regeneration. Gene Therapy 12:75-86, 2005.

Enamoto-Iwamoto M et al. Hedgehog proteins stimulate chondrogenic cell differentiation and cartilage formation. J Bone Min Res 2000; 15: 1659-1668.

Eriksen, H. A., Pajala, A., Leppilahti, J., Risteli, J. Increased content of type III collagen at the rupture site of human Achilles tendon. J Orthop Res 20:1352-1357, 2002.

Fan H, Oro A E, Scott M P, Khavari P A. Induction of basal cell carcinoma features in transgenic human skin expressing sonic hedgehog. Nature Medicine 1997: 3(7); 788-792.

Franceschi R T, Yang S, Rutherford R B, Krebsbach P H, Zhao M, Wan D. Gene therapy approaches for bone regeneration. Cells Tissues Organs 2004: 176(1-3); 95-108.

Freeman, M., and Gurdon, J. B. Regulatory principles of developmental signaling. Annu Rev Cell Dev Biol 18:515-539, 2002.

Fleming J E, Cornell C N & Muschler G F. Bone cells and matrices in orthopedic tissue engineering. Orthop Clin North Am 2000; 31: 357-374.

Frame J W. A convenient animal model for testing bone substitute materials. J Oral Surg 1980; 38: 176-180.

Gardiner, D. M., Carlson, M. R. J., and Roy, S. Towards a functional analysis of limb regeneration. Semin Cell Dev Biol 10(4):385-393, 1999.

Goetz J A, Suber L M, Zeng X & Robbins D J. Sonic hedgehog as a mediator of long-range signaling. BioEssays 2002; 24: 157-165.

Goodwin, L. O., Mason, J. M., and Hajdu, S. I. Gene expression patterns of paired bronchioloalveolar carcinoma and benign lung tissue. Ann Clin & Lab Sci 31: 369-375, 2001

Gosain A K et al. Osteogenesis in cranial defects: reassessment of the concept of critical size and the expression of TGF-β isoforns. Plast Reconsrtuct Surg 2000; 106: 360-371.

Grande D A, Breitbart A S, Mason J M, Paulino C, Laser J, and Schwartz, R E. Cartilage Tissue Engineering: Current Limitations and Solutions. Clin. Orthop. Rel. Res. 367S:S176-S185, 1999.

Grande D A et al. A dual gene therapy approach to osteochondral defect repair using a bilayer implant containing BMP-7 and IGF-1 transduced periosteal cells. Trans Orthop Res Soc 2001; 26: 294.

Grande, D. A., Mason, J. M., Light, E., and Dines, D. Stem Cells as Platforms for Delivery of Genes to Enhance Cartilage Repair. J. Bone Joint Surg Am 85A (S2):111-116, 2003.

Groeneveld E H & Burger E H. Bone morphogenetic proteins in human bone regeneration. Eur J Endocrinol 2000; 142: 9-21.

Gugala Z, Olmsted-Davis E A, Gannon F H, Lindsey R W, Davis A R. Osteoinduction by ex vivo adenovirus-mediated BMP2 delivery is independent of cell type. Gene Ther 2003:10; 1289-1296.

Gurdon, J. B., and Bourillot, P. Y. Morphogen gradient interpretation. Nature 413(6858):797-803, 2001.

Gysin R et al. Ex vivo gene therapy with stromal cells transduced with a retroviral vector containing the BMP4 gene completely heals critical size calvarial defect in rats. Gene Therapy 2002; 9: 991-999.

Henry, G., and W. L. Garner. Inflammatory mediators in wound healing. Surg Clin North Am 83:483, 2003.

Hirata K, Tsukazaki T, Kadowaki A, Furukawa K, Shibata Y, Moriish T, Okubo Y, Bessho K, Komori T, Mizuno A, Yamaguchi A. Transplantation of skin fibroblasts expressing BMP-2 promotes bone repair more effectively than those expressing Runx2. Bone 2003: 32(5); 502-512.

Hollinger J O & Kleinschmidt J C. The critical size defect as an experimental model to test bone repair materials. J Craniofacial Surg 1990; 1: 60-68.

Holmes, A., Abraham, D. J., Sa, S., Shiwen, S., Black, C. M., and Leask, A. CTGF and SMADs, maintenance of scleroderma phenotype is independent of SMAD signaling. J Biol Chem 276:10594-10601, 2001.

Hosoya M, Maruoka Y, Oda M, Asahina I, Ichinose S, Omura K. Bone with a vascular flap induced from fat tissue with the use of rhBMP-2 in rats. J Dent Res 2003: 82(8); 581-584.

Hu D & Helms J A. The role of Sonic hedgehog in normal and abnormal craniofacial morphogenesis. Development 1999; 126: 4873-4884.

Jin Q M, Anusaksathien O, Webb S A, Rutherford R B, Giannobile W V. Gene therapy of bone morphogenetic protein for periodontal tissue engineering. J Periodontol 2003: 74(2); 202-213.

Kawai S, Sugiura T. Characterization of human bone morphogenetic protein (BMP)-4 and -7 gene promoters: activation of BMP promoters by Gli, a sonic hedgehog mediator. Bone 2001: 29(1); 54-61.

Khan, P., Linkhart, B., and Simon, H-G. Different regulation of T-box genes Tbx4 and Tbx5 during limb development and limb regeneration. Dev Biol 250:383-392, 2002.

Kato M et al. Identification of Sonic Hedgehog-responsive genes using cDNA microarray. Biochem Biophys Res Comm 2001; 298: 472-478.

Kinto N et al. Fibroblasts expressing sonic hedgehog induce osteoblast differentiation and ectopic bone formation. FEBS Lett 1997; 404: 319-323.

Kramer I R, Killey H C & Wright H C. A histological and radiological comparison of the healing of defects in the rabbit calvarium with and without implanted heterogenous anorganic bone. Arch Oral Biol 1968; 13: 1095-1106.

Krebsbach P H, Gu K, Franceschi R T & Rutherford R B. Gene therapy-directed osteogenesis: BMP-7 transduced human fibroblasts form bone in vivo. Hum Gene Ther 2000; 11: 1201-1210.

Kubota K, Iseki S, Kuroda S, Oida S, Iimura T, Duarte W R, Ohya K, Ishikawa I, Kasugai S. Synergistic effect of fibroblast growth factor-4 in ectopic bone formation induced by bone morphogenetic protein-2. Bone 2002: 31(4); 465-471.

Kyllonen, H., Remitz, A., Mandelin, J. M., Elg, P., and Reitamo, S. Effect of 1-year intermittent treatment with topical tacrolimus monotherapy on skin collagen synthesis in patients with atopic dermatitis. Br J Dermatol 150:1174-1181, 2004.

Lennon D P, Haynesworth S E, Arm D M, Baber M A, Caplan A I. Dilution of human mesenchymal stem cells with dermal fibroblasts and the effect on in vitro and in vivo osteochondrogenesis. Dev Dyn 2000: 219; 50-62.

Liu, S. H., Yang, R., Al-Shaikh, R., Lane, J. M. Collagen in tendon, ligament and bone healing. A current review. Clin Orthop 318:265-278, 1995.

Loebsack A et al. In vivo characterization of a porous hydrogel material for use as a tissue bulking agent. J Biomed Mater Res 2001; 57: 575-581.

Marigo V et al. Cloning, expression and chromosomal location of SHH and IHH, two human homologues of the Drosophila segment polarity gene Hedgehog. Genomics 1995; 28: 44-51.

Markowitz D, Goff S & Bank A. A safe packaging line for gene transfer: separating viral genes on two different plasmids. J Virol 1988; 62: 1120-1124.

Martin, G. R. The roles of FGFs in the early development of vertebrate limbs. Genes Dev 12:1571-1586, 1998.

Mason J M, Grande D A, Barcia M, Pergolizzi R G, Grant R, and Breitbart A S. Expression of Human Bone Morphogenic Protein 7 in Primary Rabbit Periosteal Cells: Potential Utility in Gene Therapy for Osteochondral Repair. Gene Therapy 5:1098-1104, 1998.

Mason, J. M., Breitbart, A. S., Barcia, M., Porti, D., Pergolizzi, R. G., and Grande, D. A. Cartilage and Bone Regeneration Using Gene Enhanced Tissue Engineering. Clin. Orthop. Rel. Res. 379S:S171-S178, 2000.

Mason, J. M., Xu, H. P., Rao, S. K., Leask, A., Barcia, M., Shan, J., Stephenson, R., and Tabibzadeh, S. Lefty Contributes to the Remodeling of Extracellular Matrix by Inhibition of CTGF and Collagen mRNA Expression and Increased Proteolytic Activity in a Fibrosarcoma Model. J. Biol. Chem. 277:407-415, 2002.

Milla E et al. Poly(L-lactide) acid/alginate composite membranes for guided tissue regeneration. J Biomed Mater Res 2001; 57: 248-257.

Miller A D & Buttimore C. Redesign of retrovirus packaging cell lines to avoid recombination leading to helper virus production. Mol Cell Biol 1986; 6: 2895-2902.

Miller A D & Rosman G J. Improved retroviral vectors for gene transfer and expression. Biotechniques 1989; 7: 980-990.

Miralles G et al. Sodium alginate sponges with or without sodium hyaloronate: in vitro engineering of cartilage. J Biomed Mater Res 2001; 57: 268-278.

Miyaji H et al. Hard tissue formation on dentin surfaces applied with recombinant human bone morphogenetic protein-2 in the connective tissue of the palate. J Periodont Res 2002; 37: 204-209.

Muller, T. L., Ngo-Muller, V., Reginelli, A., Taylor, G., Anderson, R., and Muneoka, K. Regeneration in higher vertebrates: limb buds and digit tips. Semin Cell Dev Biol 10(4):405-413, 1999.

Murphy M G et al. The effects of rhBMP-2 on human osteosarcoma cells and gingival fibroblasts in vitro. J Oral Implantol 2001; 27: 16-24.

Muruve, D. A. The Innate Immune Response to Adenovirus Vectors. Gene Therapy 15:1157-1166, 2004.

Nanni L et al. SHH mutation is associated with solitary median maxillary central incisor: a study of 13 patients and review of the literature. Am J Med Genet 2001; 102: 1-10.

Nath, K. A. The tubulointerstitium in progressive renal disease. Kidney Int 54:992-994, 1998.

Ngo-Muller, V., and Muneoka, K. Influence of FGF4 on digit morphogenesis during limb development in the mouse. Dev Biol 219(2):224-236, 2000.

Nybakken K & Perrimon N. Hedgehog signal transduction: recent findings. Curr Opin Genet Develop 2002; 12: 503-51 1.

Odelber, S. J. Inducing cellular dedifferentiation: a potential method for enhancing endogenous regeneration in mammals. Semin Cell Dev Biol 13(5):335-343, 2002.

Ohsaki K, Osumi N & Nakamura S. Altered whisker patterns induced by ectopic expression of SHH are topographically represented by barrels. Dev Brain Res 2002; 137: 159-170.

Pepicelli C V, Lewis P M & McMahon A P. Sonic hedgehog regulates branching morphogenesis in the mammalian lung. Curr Biol 1998; 8: 1083-1086.

Peters H & Balling R. Teeth: where and how to make them. Trends Genet 1999; 15: 59-65.

Peterson, T. C., Pentoxifylline prevents fibrosis in an animal model and inhibits platelet-derived growth factor-driven proliferation of fibroblasts. Hepatology 17:486-493, 1993.

Rasmussen L. H., Jensen, L. T., Avnstorp, C. et al. Collagen types I and III propeptides as markers of healing in chronic leg ulcers. A noninvasive method for the determination of procollagen propeptides in wound fluid-influence of growth hormone. Ann Surg 216:684-91, 1992.

Ray, A., A. Shakya, D. Kumar, and B. K. Ray. Overexpression of serum amyloid A-activating factor 1 inhibits cell proliferation by the induction of cyclin-dependent protein kinase inhibitor p21 WAF-1/Cip-1/Sdi-1 expression. J. Immunol. 172:5006, 2004.

Reuter, M., and Kreshchenko, N. Flatworm asexual multiplication implicates stem cells and regeneration. Can J Zool/Rev Can Zool 82(2):334-356, 2004.

Roy, S., Gardiner, D. M., and Bryant, S. V. Vaccinia as a tool for functional analysis in regenerating limbs: ectopic expression of SHH. Dev Biol 218(2):199-205, 2000.

Rudert M. Histological evaluation of osteochondral defects; consideration of animal models with emphasis on the rabbit, experimental setup, follow-up and applied methods. Cells Tissue Organs 2002; 171: 229-240.

Rutherford R B, Moalli M, Franceschi R T, Wang D, Gu K, Krebsbach P H. Bone morphogenetic protein-transduced human fibroblasts convert to osteoblasts and form bone in vivo. Tissue Eng 2002: 8; 441-452.

Rutherford R B, Racenis P, Fatherazi S, Izutsu K. Bone formation by BMP-7-transduced human gingival keratinocytes. J Dent Res 2003: 82(4); 293-297.

Shang Q et al. Tissue-engineered bone repair of sheep cranial defects with autologous bone marrow stromal cells. J Craniofac Surg 2001; 12: 586-593.

Singer, M., Weckesser, E. C., Geraudie, J., Maier, C. E., Singer, J. Open finger tip healing and replacement after distal amputation in Rhesus monkey with comparison to limb regeneration in lower vertebrates. Anat Embryol 177:29-36, 1987.

Sonobe, J., Okubo, Y., Kaihara, S., Miyatake, S-I., Bessho, K. Osteoinduction by bone morphogenetic protein 2-expressing adenoviral vector: application of biomaterial to mask the host immune response. Hum Gene Ther 15:659-668, 2004.

Spinella-Jaegle S et al. Sonic Hedgehog increases the commitment of pluripotent mesenchymal cells into the osteoblastic lineage and abolishes adipocytic differentiation. J Cell Sci 2001; 114: 2085-2094.

St-Jacques B et al. Sonic hedgehog signaling is essential for hair development. Curr Biol 1998; 8: 1058-1068.

Stabler C, Wilks K, Sambanis A & Constantinidis I. The effects of alginate composition on encapsulated BTC3 cells. Biomaterials 2001; 22: 1301-1310.

Stocum, D. L. Amphibian regeneration and stem cells. Curr Top Microbiol Immunol 280:1-70, 2004a.

Stocum, D. L. Tissue restoration through regenerative biology and medicine. Adv Anat Embryol Cell Biol 176:III-VIII, 1-101, 2004b.

Strutz, F., Heeg, M., Kochsiek, T., Siemers, G., Zeisberg, M., and Miller, G. A. Effects of Pentoxifylline, Pentifylline and Gama-Interferon on Proliferation, Differentiation, and Matrix Synthesis of Human Renal Fibroblasts. Nephrol Dial Transplant 15:1535-1546, 2000.

Suzuki T et al. Regeneration of defects in the articular cartilage in rabbit temporomandibular joints by bone morphogenetic protein-2. Brit J Oral Maxillofac Surg 2002; 40: 201-206.

Torok, M. A., Gardiner, D. M., Izpisua-Belmone, J. C., and Bryant, S. V. Sonic hedgehog (shh) expression in developing and regenerating axolotl limbs. J Exp Zool 284(2): 197-206, 1999.

Tsuchida H et al. Engineered allogeneic mesenchymal stem cells repair femoral segmental defect in rats. J Orthoped Res 2003; 21: 44-53.

Valentin-Opran A et al. Clinical evaluation of recombinant bone morphogenetic protein-2. Clin Orthopaed Rel Res 2002; 395: 110-120.

Wang H-L, Carroll W J. Using absorbable collagen membranes for guided tissue regeneration, guided bone regeneration, and to treat gingival recession. Compendium 2000: 21(5); 399-412.

Wozney J M. Overview of bone morphogenetic protein. Spine 2002; 27: S2-S8.

Wright V J, Peng H, Usas A, Young B, Gearhart B, Cummins J, Huard J. BMP-4 expressing muscle-derived stem cells differentiate into osteogenic lineage and improve wound healing in immunocompetent mice. Molecular Therapy 2002: 6; 169-178.

Yoshida K et al. Enhancement by recombinant human bone morphogenetic protein-2 of bone formation by means of porous hydroxyapatite in mandibular bone defects. J Dent Res 1999; 78: 1505-1510.

Yuasa T et al. Sonic Hedgehog is involved in osteoblast differentiation by cooperating with BMP-2. J Cell Physiol 2002; 193: 225-232.

Yurek, D. M., Fletcher-Tumer, A., Moore, J., Chai, L., and Mahanthappa, N. Co-grafts of fetal ventral mesencephalon and fibroblasts expressing sonic hedgehog: effect on survival and function of dopamine grafts. Cell Transpl 10(8):665-671, 2001.

Zehentner BK, Leser U, Burtscher H. BMP-2 and sonic hedgehog have contrary effects on adipocyte-like differentiation of C3H10T1/2 cells. DNA & Cell Biology 2000: 19(5); 275-281.

Zellin G. Growth factors and bone regeneration. Swed Dent J 1998; 129: 7-65.

Zhang, Y., Nojima, S., Nakayama, H., Jin, Y., and Enza, H. Characteristics of normal stromal components and their correlation with cancer occurrence in human prostate. Oncology Reports 10:207-211, 2003.

Zuk PA et al. Multilineage cells from human adipose tissue: implications for cell-based therapies. Tissue Eng 2001; 7: 211-228.

Zuniga A, Haramis A P, McMahon A P, Zeller R. Signal relay by BMP antagonism controls the SHH/FGF4 feedback loop in vertebrate limb buds. Nature 401:598-602, 1999.

There is a large volume of work describing regeneration in lower vertebrates because they have remarkable regenerative capacity and therefore, they make excellent models for the study of factors influencing the process (Brockes, 1997; Gardiner et al., 1999). Gardiner and colleagues have been at the forefront of studies elucidating mechanisms involved in regeneration of various tissues in amphibians—animals that exhibit this regenerative capacity. Bryant, Gardiner and others demonstrated that SHH is an important player in limb regeneration in salamanders (Torok et al., 1999; Roy et al., 2000). Regeneration in higher vertebrates such as mammals, although uncommon, also occurs. For reviews of this work, see Stocum 2004a and 2004b. In this regard, mammalian liver can regenerate to some extent. In addition, digit tip regeneration has been observed in mice, monkeys, and man (Muller et al., 1999; Singer et al., 1997). But what controls these processes? Simon has observed that the regulation of two genes implicated in limb regeneration (Tbx4 and Tbx5) differs in lower and higher vertebrates (Khan et al., 2002). Ngo-Muller and Muneoka (2000) has also noted major differences in regulation of digit morphogenesis between chick and mouse. These findings suggest that much remains to be learned regarding signaling molecules as studies progress to higher vertebrates culminating in mammals. Gurdon has shown that developmental signaling can be mediated through generation of morphogen gradients (Gurdon and Bourillot, 2001) and how the supply of signal factors (such as SHH) is often the limiting step in initiating a signaling process necessary for regeneration (Freeman and Gurdon, 2002). These signaling factors are potent; only minute amounts of extracellular signaling factors are necessary to greatly influence regeneration. This suggests that supplying a morphogen such as SHH at the wound site may prove useful for enhancing regeneration if a delivery method can be identified that supplies an appropriate amount of morphogen for a suitable period of time. In support of this concept, Muneoka, Taylor and others reported that the reformation of the distal tip of the mouse limb bud is accompanied by re-expression of SHH (Muller et al., 1999). SHH is known to activate expression of a number of genes including genes in the FGF family (Zuniga et al., 1999). Members of the FGF family of signaling molecules are essential for limb outgrowth (Martin, 1998). Other work has been directed to how gene enhanced cells might be implanted into defects to regenerate various tissues in mammalian models (Mason et al., 2002; 1998; 2000; Breitbart et al., 1998; 1999a; 1999b; 2001; 2003; Grande et al., 1999; 2003; Edwards 2005).

With regard to bone regeneration, indications for bone grafting in dental and craniofacial reconstruction include bone augmentation prior to prosthetic reconstruction, fracture repair, and repair of facial bone defects secondary to trauma, tumor resection, and congenital deformities. The ideal graft material provides a source of cells capable of forming bone when suitably induced, provides the appropriate signals to induce bone formation (an osteoinductive environment), and provides a scaffold for new bone formation (an osteoconductive environment).

The regulation of bone metabolism is mediated by both systemic and local factors (Zellin, 1998). Of these, the bone morphogenetic proteins (BMPs) and Sonic hedgehog (SHH) appear to be involved in the formation of new bone, both embryologically and in the repair of fractures.

BMPs are a family of morphogens that regulate bone formation and promote fracture healing, in part by stimulating the differentiation of noncommitted precursor cells into osteoblasts (Ebara and Nakayama, 2002). Studies involving the use of exogenously administered recombinant BMP-2 and BMP-7 to induce bone regeneration have generally been promising in lower animals (Yoshida et al., 1999; Miyaja et al., 2002; Suzuki et al., 2002). A number of recent articles have reviewed the potential for BMP delivery in human bone regeneration (Wozney, 2002; Groeneveld and Burger, 2000; Valentin-Opran et al., 2002).

Sonic hedgehog (SHH), a 45 kDa vertebrate homolog of the Drosophila segment polarity gene (hedgehog) and a member of the Hedgehog gene family (Sonic, Desert, and Indian hedgehog), is a key protein involved in craniofacial morphogenesis. SHH causes differentiation of pluripotent mesenchymal stem cells into the osteoblastic lineage by upregulating BMPs via Smad signaling (Spinella-Jaegle et al., 2001). SHH also induces cell proliferation in a tissue-specific manner during embryogenesis via the regulation of epithelial-mesenchymal interactions (e.g. hair follicles [St-Jacques et al., 1998] and teeth [Peters and Balling, 1999; Dassule et al., 2000]).

The importance of SHH to craniofacial morphogenesis has been demonstrated in experiments with SHH-null mutant mice in which the first branchial arch, which gives rise to both the mandible and maxilla, fails to form (Chiang et al., 1996). Moreover, mutations in the human SHH gene have been shown to cause holoprocencephaly (Hu and Helms, 1999), a developmental field defect in which the cerebral hemispheres fail to separate into distinct halves. Associated anomalies include hypotelorism, midline cleft lip/palate, proboscis-like nasal structures, and premaxillary agenesis. Mutations of the SHH gene have been identified in the rare dental anomaly, solitary median maxillary central incisor (Nanni et al., 2001). Additionally, excess SHH leads to a mediolateral widening of the frontonasal process and hypertelorism (Hu and Helmes, 1999).

SHH increases the commitment of pluripotential mesenchymal cells into the osteoblastic lineage (Spinella-Jaegle et al., 2001; Kinto et al., 1997) by stimulating the expression of a cascade of downstream genes involved in bone development (Kato et al., 1997; Nybakken and Perrimon, 2002; Bitgood and McMahon, 1995). Transduction of an SHH-coding adenovirus into mouse embryo induces the ectopic expression of BMP-4, Patched-1, Patched-2, and Gli1 (Ohsake et al., 2002). Ectopic bone formation can be induced in athymic mice by transplantation of SHH-transfected chicken fibroblast cells (Kinto et al., 1997). Implantation of SHH-enhanced chicken embryo-derived dermal fibroblasts into nude mice results in ectopic cartilage and bone formation (Enamoto-lwamoto et al., 2000). However, intramuscular transplantation of SHH protein alone does not induce bone formation (Yuasa et al., 2002). This suggests that either the in vivo half-life of SHH is too short to establish the gradient required of SHH to exert its effect (Goetz et al., 2002), or that SHH must function in concert with other downstream factors involved in bone regeneration.

Following injury, many tissues in the body are capable only of repair, often characterized by fibroblast overgrowth to “fill” the void left by the injury with scar tissue, rather than true regeneration of tissue to its previous fully functional state. Such scar tissue formation is problematic due to reduced function and because once this filler tissue is generated, it interferes with regeneration of the fully functional tissues. This occurs in many of the body's tissues and is plainly exemplified in nerve regeneration where the body is often quite capable of slow re-growth of neural networks, but the more rapid formation of scar tissue ultimately prevents proper innervation of the distal organs. In effect, the problem to be overcome is one of evolution. To optimize short-term survival, nature has evolved to select repair mechanisms resulting in a “quick fix” to rapidly patch injuries, but unfortunately this patch is mainly scar tissue. Simply put—there is a competition to repair injury through fibrosis vs. regeneration; fibrosis is a faster process and thus wins the race. Biological advances are needed to improve tissue regeneration protocols for congenital or induced (e.g., from injury) tissue defects by overcoming nature's predilection to rapidly fill injuries with scar tissue while simultaneously optimizing the speed and quality of regeneration of multiple tissue and organ systems. The present invention addresses that need.

### SUMMARY OF THE INVENTION

Accordingly, the inventor has developed improvements in methods for repairing tissue defects using a matrix with embedded cells. The improvements include the use of cells that express recombinant sonic hedgehog, and a matrix that comprises alginate and collagen type 1.

Thus, the invention is directed to mammalian cells comprising a recombinant sonic hedgehog (SHH) gene such that a recombinant SHH protein can be expressed by the cell. In these embodiments, the cell is a stem cell, a fat-derived fibroblast-like cell, a gingival cell, or a periosteum cell, and the SHH protein amino acid sequence is at least 90% homologous to SEQ ID NO:1 or SEQ ID NO:2.

The invention is also directed to matrices suitable for applying to a tissue defect, where the matrices comprise alginate and collagen type I.

The invention is additionally directed to tissue regeneration compositions comprising the cells described above in a biocompatible matrix.

The invention is further directed to methods of regenerating tissue at the site of a tissue defect in a mammal, where the tissue is bone, dermis, nervous tissue, or tendon. The methods comprise combining the cell described above with the matrix described above, then applying the cell—matrix combination to the defect for a time sufficient to regenerate the tissue.

Additionally, the invention is directed to methods of regenerating tissue at the site of a tissue defect in a mammal. The methods comprise combining a mammalian cell with a vector, embedding the cell-vector combination in a matrix suitable for applying to a tissue defect, then applying the matrix-cell-vector combination to the tissue defect for a time sufficient to regenerate the tissue. In these methods the cell-vector combination is not expanded in culture before the cell-vector-matrix combination is applied to the tissue defect.

### BRIEF DESCRIPTION OF THE DRAWINGS

FIG. 1 is a diagram of the retroviral expression vectors used in the Example. Constructs are based on the LN series of vectors containing the selectable neomycin-resistance gene driven by the 5′ murine leukemia virus retroviral LTR. In the retroviral vector plasmid pLNB-SHH, the SHH cDNA (isolated from human fetal lung tissue) was cloned as a HindIII/Clal fragment replacing the BMP-7 HindIII/Clal fragment in plasmid pLNB-BMP-7. The rat β-actin enhancer/promoter was chosen to drive expression of SHH. These retroviral vectors are generated as amphotropic retroviral vector particles from PA317 cells. Sizes of genomic length RNA and mRNA are indicated. (LTR: long terminal repeat, Neo^{r}: neomycin-resistance gene; β-act: rat-actin promoter; SHH: Sonic hedgehog gene).

FIG. 2 is a photograph of an electrophoresed agarose gel with products of a reverse transcriptase-polymerase chain reaction (RT-PCR) analysis of total RNA isolated from periosteal-derived cells. Total RNA was isolated from 1×10⁶ transduced cells. Oligonucleotide PCR primers NS145 5′aaaaagcttgggcgagatgctgctgctggcgagatgtct 3′ (SEQ ID NO:4)(forward primer in 5′ coding sequence of SHH) and NS239 5′ ccctttttctggagactaaataaaatc 3′ (SEQ ID NO:13)(reverse primer downstream of the SHH gene in the viral vector) were used to amplify a 1446 bp fragment encompassing the 5′ end of SHH and flanking vector sequence encoded specifically by LNB-SHH at an annealing temperature of 70° C. (30 cycles). GAPDH primers NS159 (5′ ggtcatccctgagctgaacg 3—SEQ ID NO:14) and NS160 (5′ ttcgttgtcataccaggaaat 3—SEQ ID NO:15) at an annealing temperature of 55° C. (30 cycles) were used as control of RNA quality. Other controls included a no template control. From left to right, the groupings of cell lines are (a) gingival fibroblasts, (b) fat-derived stem cells, and (c) periosteal-derived cells. The order of the lanes is the same in each group. Lanes: (1) 1 kb DNA marker. (2) LNB-SHH-transduced cells amplified with SHH-specific (NS145) and retroviral vector-specific (NS239) primers. The expected 1446 kb SHH transcript is generated. (3) LNCX control-transduced cells amplified with SHH-specific (NS145) and retroviral vector-specific (NS239) templates. No transcript is generated. (4) GAPDH control for RNA integrity, LNB-SHH-transduced cells, using GAPDH-specific primers NS159 and NS160. The expected 294 bp GAPDH PCR product was generated. (5) GAPDH control for RNA integrity, LNCX-control-transduced cells, using GAPDH-specific primers NS159 and NS160. The expected 294 bp GAPDH PCR product was generated. (6) No template control (water lane) control. No transcript is generated.

FIG. 3 are micrographs of results of a histological study of alginate/type I collagen/periosteal-derived cells composite bone graft material. In order to assess the viability of cells in this composite bone graft material, composites of alginate/type I collagen/periosteal-derived cells were prepared and submitted for histological sectioning immediately after assembly (time 0) and after 7 days in culture. Panel a shows alginate/type I collagen/periosteal-derived cell composite graft with 2×10⁶ cells/450 μl matrix construct, time 0. Panel B shows a similar graft after 7 days culture in vitro. Hematoxylin and eosin stain. Original magnification 100× (inset 200×).

FIG. 4 shows a radiographic analysis of calvarial bone regeneration at 6 weeks. Full thickness 8 mm cranial bone defects (frontal and parietal bones, four defects per rabbit) were created in adult male New Zealand White rabbits using a trephine bur. The surgically created defects were restored with the selected transduced cells or the corresponding controls in an alginate/type 1 collagen matrix. Dosing was accomplished at the cell number level (2×10⁶ cells per defect). The experimental groups comprised allogenic gingival fibroblasts, periosteal and fat-derived stem cells transduced with the replication-incompetent SHH retroviral vector (LNB-SHH) and control vector (LNCX). Additional controls included alginate/collagen matrix alone and empty defects. After 6 weeks, the animals were killed and post-mortem radiographs were taken. Six calvarial defects were analyzed for each experimental group. As demonstrated in this sample radiograph, significantly more bone regeneration is evident in the SHH-enhanced gingival fibroblasts (upper right) compared to the three controls.

FIG. 5 is micrographs showing experimental results of a histologic assessment of bone regeneration at 6 weeks. At 6 weeks post defect restoration, the animals were killed. The defect sites were identified visually and submitted for histologic examination. Six calvarial defects were analyzed for each experimental group. After decalcification for 3 days, the tissue was embedded in paraffin and 4 m sections closest to the center of the defect, showing the full diameter of the defects, were cut and stained with hematoxylin and eosin. Composite photomicrographs were assembled from these histological sections. Panel a shows an unrestored empty defect. Only a thin band of fibrous connective tissue is present in the defect space. Panel b shows a bone defect treated with matrix alone. The matrix has preserved the thickness of the defect space. New bone formation is minimal. Panel c shows a bone defect treated with matrix plus control-transduced fat-derived stem cells. New bone formation is minimal. Similar results were observed with the control-transduced periosteal-derived cells and control-transduced gingival fibroblasts (data not shown). Panel d shows a bone defect treated with matrix plus SHH gene-enhanced periosteal-derived cells. Thin trabeculae of new bone are identified primarily at the surgical margins. Panel e shows a bone defect treated with matrix plus SHH gene-enhanced fat-derived stem cells. Relatively thick trabeculae of new bone are present, but this new bone is not evenly distributed throughout the defect site. Panel f shows a bone defect treated with matrix plus SHH gene-enhanced gingival fibroblasts. A significant amount of new bone is present throughout the defect space. Hematoxylin and eosin stain. Original magnification 4×.

FIG. 6 is a micrograph of a histologic assessment of bone regeneration in SHH gene-enhanced gingival fibroblasts at 6 weeks. This close up photomicrograph demonstrates significant new bone formation at 6 weeks in the defects restored with SHH-gene-enhanced gingival fibroblasts in our alginate/type I collagen matrix (2×10⁶ cells per 8 mm calvarial defect). The amorphous, purple material represents remaining matrix. Hematoxylin and eosin stain. Original magnification 40×.

FIG. 7 is micrographs showing experimental results of a histologic assessment of bone regeneration at 12 weeks. After 12 weeks, the animals were killed. The defect sites were identified visually and submitted for histologic examination. Six calvarial defects were analyzed for each experimental group. After decalcification for 3 days, the tissue was embedded in paraffin, and 4 m sections closest to the center of the defect, showing the full diameter of the defects, were cut and stained with hematoxylin and eosin. Composite photomicrographs were assembled from these histological sections. Panel a shows an unrestored empty defect. A thin band of fibrous connective tissue and minimal new bone is present in the defect space. Panel b shows a bone defect treated with matrix alone. While the matrix has preserved the thickness of the defect space, new bone formation is minimal. Panel c shows a bone defect treated with matrix plus control-transduced gingival fibroblasts. New bone formation is minimal. Similar results were observed with the control-transduced fat-derived stem cells and control-transduced periosteal-derived cells (data not shown). Panel d shows a bone defect treated with matrix plus SHH gene-enhanced periosteal-derived cells. Thin trabeculae of new bone are identified, primarily at the margins. Panel e shows a bone defect treated with matrix plus SHH gene-enhanced fat-derived stem cells. Similar to what was noted at 6 weeks, relatively thick trabeculae of new bone are present, but this new bone is not evenly distributed throughout the defect site. Panel f shows a bone defect treated with matrix plus SHH gene-enhanced gingival fibroblasts. A significant amount of new bone is present throughout the defect space. Similar to the findings noted at 6 weeks, the SHH gene-enhanced gingival fibroblasts afforded the best-dispersed overall bone regeneration. Hematoxylin and eosin stain. Original magnification 4×.

FIG. 8 is a micrograph of a histologic assessment of bone regeneration in SHH gene-enhanced gingival fibroblasts at 12 weeks. Close up photomicrograph, demonstrating significant new bone formation at 12 weeks in the defects restored with SHH—gene-enhanced gingival fibroblasts in our alginate/type I collagen matrix (2×10⁶ cells per 8 mm calvarial defect). Bone formation is evident in direct continuity with the matrix. Bone marrow is also identified. In areas where new bone formation was not complete (lower left), the remaining matrix had a lower density of cells. Hematoxylin and eosin stain. Original magnification 40×.

FIG. 9 is a graph of experimental results showing bone regeneration in calvarial defects at 6 and 12 weeks. Histologic slides, obtained from defect sites at 6 and 12 weeks, were digitized and the total two-dimensional amount of new bone in the surgically created defects was quantitated. Briefly, digitized composite photomicrographs were analyzed on an IBM PC running Windows 98 with Adobe Photoshop 6.0. The mineralized area of the defects in the digitized radiographs was identified by the value of the pixel in the image. The percentage of area of mineralized tissue within the defect size was determined. Data was analyzed using ANOVA followed by pairwise comparison. A ‘P’-value of less than 0.05 was considered statistically significant. In all cases, SHH gene enhancement of cells resulted in statistically significant differences (P<0.05) compared to controls.

### DETAILED DESCRIPTION OF THE INVENTION

Gene-based therapies involve delivering a specific gene to target tissue with the goal of changing the phenotype or protein expression profile of the recipient cell. A primary goal of gene-enhanced tissue engineering is to recapitulate the stages of tissue regeneration to produce tissue that is indistinguishable from normal host bone. In a step toward that goal, the present invention utilizes a gene-enhanced tissue-engineering approach to develop a bone grafting material that is effective at regenerating both small and large defects of several different tissues.

With the present invention, improved regeneration is achieved through the manipulation of three components: a regeneration inducing agent, a cellular component, and a biodegradable matrix. The matrix is versatile enough for use in defects of various tissues to contain the cells and inducing agent. Morphogenic proteins are also used to cause various fibroblast-like cells to transdifferentiate into multiple cell types needed for regeneration of adult tissue defects. Surprisingly, fibroblast-like cells derived from different tissues (periosteum, gingiva, and fat) all effectively regenerated bone, but only when genetically enhanced with sonic hedgehog (SHH) (see Example, published as Edwards et al., 2005).

Thus, the invention is directed to mammalian cells comprising a recombinant sonic hedgehog (SHH) gene such that a recombinant SHH protein can be expressed by the cell. In these embodiments, the cell is a stem cell, a fat-derived fibroblast-like cell, a gingival cell, or a periosteum cell, and the SHH protein amino acid sequence is at least 90% homologous to SEQ ID NO:1 (a human SHH amino acid sequence) or SEQ ID NO:2 (a rat SHH amino acid sequence).

It is believed that SHH first primes cells to differentiate and then local cues in the wound environment determine which tissue the cells should differentiate into. This phenomenon was observed in regeneration of rabbit osteochondral defects where the same cells, transfected with BMP-7, transdifferentiated to regenerate both bone and cartilage in orthotopically correct locations (Mason et al., 2000).

SHH is a potent morphogen that drives target cell differentiation into osteoblasts (Zehentner et al., 2000; Spinella-Jaegle et al., 2001). SHH has the advantage that it is a secreted, diffusible morphogen that can act on non-enhanced cells at both proximal and distal sites. SHH regulates expression of a cascade of genes involved in bone development (Hu and Helmes, 1999) including Gli-1, BMP-4, BMP-7 (Kawai and Sugiura, 2001), BMP-2 (Fan et al., 1997) and FGF-4 (Zuniga et al., 1999). Because SHH induces expression of multiple BMPs, it is believed to stimulate formation of a desirable mixture of homo and heteromeric BMPs, mimicking normal bone matrix (Wozney, 2002; Franceschi et al., 2004) and resulting in beneficial synergistic effects in bone regeneration (Kubota et al., 2002). The results of the experiments described in the Example bears this out.

The invention cell is preferably a stem cell. However, other cell types are also useful for repairing defects. Lennon et al., 2000, reported that a 50% mixture of mesenchymal stem cells (MSC) and fibroblasts demonstrated no reduced effects on quantifiable osteogenic parameters compared to pure MSC in various in vitro and in vivo assays. This finding coupled with the data provided in the Example, which used populations of non-purified fibroblast-like cells from various tissues, demonstrates that complex, laborious, and expensive purification procedures are not necessary to obtain full regenerative potential of various cell sources for bone and osteochondral tissue regeneration when cells are SHH supplemented. Fibroblasts isolated from fat, dermis, and gingiva (all relatively abundant cell sources) potentially have osteogenic potential (Hirata et al., 2003; Jin et al., 2003; Gugala et al., 2003; Hosoya et al., 2003; Dragoo et al., 2003; Rutherford et al., 2002; 2003). However, morphogen supplementation does not cause all cell types (i.e. keratinocytes) to transdifferentiate into osteoblasts (Rutherford et al., 2003). Thus, certain mammalian cell types can be induced to dedifferentiate to progenitor cells when stimulated with appropriate signals (see also Odelber, 2002).

Based on the above discussion and the results in the Example, the dogma that rare populations of stem cells existing in various tissues must be used to regenerate different tissues and that these rare cells must be recruited to the wound by unknown mechanisms to elicit wound repair is incorrect. Although stem cells are useful for regenerating tissue, abundant fibroblast-like cells from gingiva, periosteum, or fat are also capable of regenerating bone (See Example). These are not stem cells because they only regenerate bone when genetically enhanced with the SHH gene. However, they can effectively respond to morphogen signaling to regenerate tissues. The inventors believe that the tissues that the cells differentiate into are determined by other local factors in the wound environment.

Thus, the cell can alternately be a gingival cell; in still other embodiments, the cell is a periosteum cell. In the most preferred embodiments, the cell is a fat-derived fibroblast-like cell. Fat is an easily harvested source of these fibroblast-like cells. Fat is also generally abundant, largely expendable, renewable, and the easiest donor tissue to obtain with minimal scarring and donor site trauma. In addition, the fibroblast-like cells can be obtained by simple and rapid processing of fat. No extensive purification schemes or ex vivo expansion of cells is required.

The cell is preferably a human cell. Most preferably, the cell is preferably autologous (i.e., from the same individual to be treated with the cells) because no exogenous disease transmission or immune rejection issues should arise. However, allogenic (from a different animal, preferably in the same species) cells can also be used (Example, Mason et al., 2000; Breitbart et al., 2001; Arinzeh et al., 2003).

As used herein, SHH is a mammalian protein that has at least 90% amino acid homology to SEQ ID NO:1 or SEQ ID NO:2 and is effective in improving regeneration of a bone, dermis, nervous tissue, or tendon defect when the cells of the present invention recombinantly express the SHH and are transplanted into the defect in a suitable matrix (e.g., the alginate/collagen matrix described below).

The SHH protein is preferably a wild-type SHH protein. However, genes encoding mutants of wild-type SHH proteins can also be utilized in the present invention provided the mutant SHH protein is also effective in improving tissue regeneration. The skilled artisan could identify numerous mutants of a wild-type SHH protein that would be expected to be useful for the present invention by, e.g., identifying amino acid residues that are not conserved among species, or by making hybrids of SHH proteins from two different species.

The SHH gene preferably encodes an SHH protein from the same species as the intended recipient. However, the present invention also encompasses mammalian cells where the transfected SHH gene is from a different species as the cell and/or the intended recipient, since SHH is highly conserved among mammals

Mammalian SHH proteins are preferred; in the most preferred embodiments, the SHH protein is a human protein.

The cell can further comprise a second recombinant gene such that the second recombinant gene can be expressed by the cell. These embodiments are not limited to any particular second recombinant gene. Nonlimiting examples of useful second recombinant genes are those encoding a fluorescent protein, a factor that enhances nerve regeneration, a transcriptional regulator, a lefty protein, a platelet derived growth factor, a transforming growth factor, a fibroblast growth factor, an insulin-like growth factor, a bone morphogenic protein (BMP), a parathyroid hormone, a parathyroid hormone-like related protein, a growth hormone, a vascular endothelial growth factor, an Oct4, a Nanog, a Runx2/Cbfal, an Osterix, a Sox9, a DLX2-6, a Msx, a bone sialoprotein, a dentin sialophosphoprotein, a matrix Gla protein, an osteopontin, or a soluble BMP receptor.

Where the defect in an intended recipient is in a nervous tissue, a preferred second recombinant gene encodes a factor that enhances nerve regeneration. The expression of such a factor could enhance the ability of SHH to assist cells to differentiate into neurons or other brain specific cell types (Yurek et al., 2001; Craven et al., 2004).

Nonlimiting examples of useful factors that enhance nerve regeneration are nerve growth factor, neurotrophin-3, neurotrophin 4/5, leukemia inhibitory factor, brain-derived neurotrophic factor, ciliary neurotrophic factor, glial cell line-derived neurotrophic factor, neurturin, persephin, and artemin.

Where the defect in an intended recipient is bone, a useful second recombinant gene encodes a bone morphogenic protein (BMP), most preferably BMP-7.

Another important second recombinant gene encodes lefty, which acts in opposition to TGF-β to inhibit fibrosis (Mason et al., 2002). Excessive collagen accumulation is a hallmark of fibrosis, which can interfere with subsequent tissue regeneration. However, collagen synthesis as a process must be approached carefully with regard to tissue regeneration. Two important considerations are involved. First, some level of collagen synthesis is beneficial for regeneration of various tissues and it is not desirable to reduce levels to the point where it is detrimental to regeneration. Type III collagen often is the first type of collagen to form in areas of tissue growth and regeneration. Subsequently, type I collagen often replaces the type III. Type III collagen levels are increased in healing leg ulcers and in the repair site of ruptured Achilles tendons (Rasmussen et al., 1992; Eriksen et al., 2002; Liu et al., 1995).

SHH is a very short-lived protein and dosing with such potent factors is important for establishing an appropriate protein gradient optimal for regeneration (Gurdon and Bourillot, 2001). It is extremely difficult to establish proper protein gradients through delivery of large boluses of short-lived proteins. Therefore, delivery of SHH in the present invention is accomplished by genetic enhancement of cells with genes encoding SHH via a vector.

The invention is not narrowly limited to any particular type of vector. In some embodiments, the recombinant SHH gene is transfected into the cell as part of a naked DNA vector. Preferably, the recombinant SHH gene is transfected into the cell as part of a viral vector. Preferred viral vectors include adenovirus, retrovirus, adeno-associated virus and herpes simplex virus vectors. More preferably, the viral vector is a replication-incompetent retrovirus. Most preferably, the viral vector is an adenovirus.

Adenoviral vectors are preferred at this time for practical purposes. They are easily produced in large amounts and can be lyophilized and stored at room temperature for ease of shipment. They also express the delivered genes maximally for only 1 to 2 weeks; enough time for the expressed SHH to transdifferentiate the fibroblast-like cells to promote regeneration. Long term expression of SHH is not desirable due to the possibility of unforeseen negative effects. Once the tissue is regenerated, continued expression of SHH is not desirable, therefore, the short term expression inherent with use of adenoviral vectors is a useful characteristic.

Adenoviral vectors are transient in the cell because the vectors are episomal and the host cell eventually recognizes the adenoviral vector genome as foreign and neutralizes it. However, two weeks of expression is generally sufficient to initiate the morphogen gradients and trigger transdifferentiation. Two week expression is an advantage for situations where expression of transgenes for this limited period of time is desirable and where avoidance of use of integrating vectors is considered advantageous. Adenoviral vectors have the disadvantage that they often trigger an immune response to the transduced cells due to adenoviral proteins expressed along with the transgene. The fact that bone, brain, and tendon defects may be immune-privileged sites (we have repeatedly used allogenic cells to repair bone defects without observing deleterious immune responses) or that the matrix material is masking foreign antigens (Sonobe et al., 2004) lessens the possibility of a deleterious immune reaction to the adenoviral vectors in a recipient of cells infected with the adenoviral vectors.

Useful control elements directing the expression of the SHH such as promoters and enhancers can be determined for any particular purpose by the skilled artisan without undue experimentation. A commonly used element is the Cytomegalovirus (CMV) enhancer/promoter, which directs high levels of expression, particularly when used in an adenoviral vector, where very high expression occurs. However, the amount of morphogen present is a critical factor and too much morphogen could be toxic (Gurdon and Bourillot 2001). Toxicity to transduced target cells that overexpress BMP-7 using the strong CMV promoter in an osteochondral defect model has also been observed (Mason et al., 1998). Use of the weaker β-actin enhancer/promoter solved the toxicity problem and resulted in excellent bone formation in subsequent experiments. Consequently, preferred enhancers and promoters are those that direct an intermediate level of expression such as the β-actin enhancer/promoter.

Where the cell comprises a second recombinant gene as described above, that second recombinant gene is also preferably transfected into the cell as part of a viral vector. More preferably, that viral vector is an adenovirus, a retrovirus, an adeno-associated virus or a herpes simplex virus; even more preferably a replication-incompetent retrovirus. Most preferably, the viral vector is an adenovirus. It is also preferred that both the recombinant SHH gene and any second recombinant gene is transfected into the cell as part of an adenovirus. The recombinant SHH gene and the second recombinant gene can be transfected into the cell as part of the same adenovirus or part of different adenoviruses.

Preferably, the recombinant SHH protein is constitutively expressed in the cell. However, the invention also encompasses cells where the recombinant SHH protein (and/or the second recombinant gene, if present) is under the control of an inducible promoter.

It is also preferred that the cell eventually loses the recombinant SHH gene, e.g., if the recombinant SHH gene is part of an adenoviral vector. The recombinant SHH gene can also be on another episomal vector or an episomal nucleic acid like a plasmid that is eventually lost from the cell. Preferably, the recombinant SHH gene is operably linked to a β-actin enhancer and promoter.

Preferably, the vector is a replication-incompetent retrovirus, the recombinant SHH gene is operably linked to a β-actin enhancer and promoter, and the mammalian cell is a gingival cell or a periosteum cell. More preferably, the vector is a replication-incompetent retrovirus, the recombinant SHH gene is operably linked to a β-actin enhancer and promoter, and the mammalian cell is a fat-derived fibroblast-like cell. Even more preferably, the vector is an adenovirus, the recombinant SHH gene is operably linked to a β-actin enhancer and promoter, and the mammalian cell is a gingival cell or a periosteum cell. Most preferably, the vector is an adenovirus, the recombinant SHH gene is operably linked to a β-actin enhancer and promoter, and the mammalian cell is a fat-derived fibroblast-like cell.

When the cells described above are to be utilized for tissue engineering, e.g., to regenerate tissue at the site of a defect in bone, dermis, nervous tissue or tendon, the cell is preferably part of a matrix suitable for applying to a tissue defect. The most preferred matrices comprise alginate and collagen type I, such as used in the Example.

The invention is also directed to matrices suitable for applying to a tissue defect, where the matrices comprise alginate and collagen type I.

The ideal scaffold for tissue engineering should be relatively easy to handle, allow for incorporation of cells, provide a three-dimensional scaffold to hold the space of the defect, allow for the free diffusion of cells and growth factors, establish a vascular bed within the first few days after implantation to ensure survival of the implanted cells, induce a minimal inflammatory response and be ultimately biodegraded. A composite matrix of alginate and collagen has these ideal properties and are useful for bone regeneration (see Example) and regeneration of other tissues in various orthotopic sites.

The alginate/collagen type I matrix has excellent features as a versatile matrix: it is malleable, adaptable to mold to any wound; it is biodegradable and biocompatible, showing no negative effects from breakdown products as sometimes observed with materials such as polyglycolic acid (PGA) and polylactic acid (PLA); cells do not have to “seed” onto matrix materials which also requires cell culturing for extended periods of time; the percentage of alginate and collagen in the matrix can be easily customized for different tissues should it be necessary; this matrix can be formulated to be an injectable if necessary; there are many types of alginate with different pore sizes for built in flexibility for porosity requirements in different tissues; for practical purposes, alginate (from seaweed) and collagen type I are readily commercially available and in plentiful supply; and alginate and collagen type I are already approved for clinical use.

Purified bovine collagen is biocompatible and has been shown to promote regeneration of bone defects in a various models (Cornell, 1999). However, a collagen-based system alone does not afford a matrix with the necessary firmness and did not have the requisite strength to hold the defect space. Moreover, collagen gels tend to rapidly contract and lose their shape and consistency (Diduch et al., 2000). Therefore, the use of an alginate/collagen I based matrix is preferred. In these matrices, the alginate provides a structural mesh around the cells and collagen, and the collagen increases the osteoconductive nature of the scaffold and also helps distribute the cells evenly throughout the porous alginate scaffold (Wang and Carroll, 2000).

Alginate is a biodegradable polysaccharide composed of mannuronic and guluronic acid units. The porous nature of alginate gels allows for the migration of cells and regulatory proteins inside the network (Stabler et al., 2001). The matrices of the present invention are not limited to any of the many sources and grades of alginate; the skilled artisan could select a suitable alginate for any particular purpose without undue experimentation. A preferred alginate is from Macrocystitis pyrifera (kelp) with a medium viscosity (3000 CPS) and composed of 61% mannuronic and 39% guluronic acid (M/G ratio of 1.56) and a molecular weight of ˜100,000 D.

The alginate in the invention matrices is preferably between about 0.5% and about 3% (w/v) and the collagen type I is between 0.1 mg/ml and 5 mg/ml. More preferably, the alginate is about 1% (w/v) and the collagen type I is about 1.2 mg/ml. Medium viscosity alginate comprising mannuronic acid and guluronic acid at a ratio of between 1 and 2 is most preferred.

In some applications, such as when it is particularly undesirable to expose an internal defect in a recipient of the cell-matrix combination, the cell-matrix combination is injectable.

Most preferably, the matrix further comprises the recombinant cells expressing an SHH gene as described above.

The number of cells initially seeded in defects is an important parameter in tissue regeneration (Awad et al., 2000, Wright et al., 2002; Gysin et al., 2002). In the work described in the Example, a seeding number of 2×10⁶ cells/implant (˜5×10⁶ cells/ml by volume) was used. However, to optimally regenerate bone, a greater number is preferred. See Example,. where implanting the matrix at that density lead to some spotty regions of the implant that did not rapidly regenerate bone and that were acellular. Thus, higher densities are useful in various applications. The ideal cell density for any particular application can be determined by the skilled artisan without undue experimentation. Preferably, the mammalian cells present in the matrix are at a concentration of between 0.2×10⁷/ml and 10×10⁷/ml. Most preferably, the mammalian cells are present in the matrix are at a concentration of about 1×10⁷/ml

These matrices can also usefully comprise a compound that improves regeneration of the tissue. Non-limiting examples of compounds that improve regeneration are an antibiotic, an antifibrotic agent, a factor that enhances nerve regeneration, a transcriptional regulator, a platelet derived growth factor, a fibroblast growth factor, an insulin-like growth factor, a bone morphogenic protein (BMP), a parathyroid hormone, a parathyroid hormone-like related protein, a growth hormone, a vascular endothelial growth factor, a transforming growth factor, an Oct4, a Nanog, a Runx2/Cbfal, an Osterix, a Sox9, a DLX2-6, a Msx, a bone sialoprotein, a dentin sialophosphoprotein, a matrix Gla protein, an osteopontin, or a soluble BMP receptor. In some preferred embodiments, the compound is a BMP.

Preferably, the compound incorporated into the matrix that improves regeneration is an antifibrotic agent. preferred examples include pentifylline (PTF) or pentoxifylline (PTX). See Duncan et al., 1995; Peterson, 1993; Chen et al., 1999. Aside from their antifibrotic activity, these substances also greatly reduce fibroblast proliferation and collagen type I synthesis in renal fibroblasts. The antifibrotic activity is believed to be mediated through inhibition of fibroblast growth factor 2 (FGF2) expression (Strutz et al., 2000).

The compound incorporated into the matrix that improves regeneration can be combined with the matrix as the compound itself, or, where the compound is a protein, can be expressed by a recombinant cell expressing the compound. Thus, the matrices described above that comprise mammalian cells expressing SHH can further comprise second recombinant cells where the second recombinant cells comprise a second transgene encoding a compound that improves regeneration of the tissue. The compound is preferably a factor that enhances nerve regeneration, a transcriptional regulator, a lefty gene, a platelet derived growth factor, a transforming growth factor, a fibroblast growth factor, an insulin-like growth factor, a bone morphogenic protein (BMP), a parathyroid hormone, a parathyroid hormone-like related protein, a growth hormone, a vascular endothelial growth factor, an Oct4, a Nanog, a Runx2/Cbfal, an Osterix, a Sox9, a DLX2-6, a Msx, a bone sialoprotein, a dentin sialophosphoprotein, a matrix Gla protein, an osteopontin, or a soluble BMP receptor. More preferably, the second transgene encodes a lefty protein, to reduce fibrosis.

Lefty is a protein in the TGF-β superfamily which plays a role in opposition to TGF-β (Mason et al., 2002). Expression of lefty from fibroblastic cells caused them to lose their ability to deposit collagen in vivo. Lefty also inhibits the TGF-β mediated promoter activity of connective tissue growth factor (CTGF), which induces proliferation of fibroblasts and collagen synthesis (Holmes et al., 2001).

The invention is also directed to tissue regeneration compositions comprising the mammalian cells described above that express SHH, in a biocompatible matrix. These tissue regeneration compositions are useful for applying to a tissue defect to regenerate the tissue.

Preferably, the biocompatible matrix comprises alginate and collagen type I. The cell in these compositions is preferably a human cell. Additionally, the cell can usefully further comprise a second recombinant gene such that the second recombinant gene can be expressed by the cell. The tissue regeneration compositions can also comprise second recombinant cells, where the second recombinant cells comprise a second transgene encoding a compound that improves regeneration of the tissue. The matrix can also comprise a compound that improves regeneration of the tissue.

In preferred tissue regeneration compositions, the vector is an adenovirus, the recombinant SHH gene is operably linked to a β-actin enhancer and promoter, and the mammalian cell is a gingival cell or a periosteum cell. In the most preferred embodiments, the vector is an adenovirus, the recombinant SHH gene is operably linked to a β-actin enhancer and promoter, and the mammalian cell is a fat-derived fibroblast-like cell. a gingival cell.

The invention is also directed to methods of regenerating tissue at the site of a tissue defect in a mammal, where the tissue is bone, dermis, nervous tissue, or tendon. The methods comprise combining the cell described above with the matrix described above, then applying the cell-matrix combination to the defect for a time sufficient to regenerate the tissue. Preferably, the tissue is bone. The cell is preferably a stem cell, a fat-derived fibroblast-like cell, a gingival cell, or a periosteum cell; more preferably, the cell is a gingival cell or a periosteum cell. Most preferably, the cell is a fat-derived fibroblast-like cell. The cell is also preferably a human cell.

Preferably, the mammal in these embodiments is a human. It is also preferred that the cell is from the same species of the mammal, more preferably from the mammal. Most preferably, the cell is a human cell, the mammal is a human, and the SHH protein is a wild-type human SHH protein having at least 95% amino acid homology to SEQ ID NO:1.

The cell-matrix combination can be incubated before application to the defect in order to expand the transgenic cell population before implantation. However, this incubation is often not necessary, and the cell-matrix combination is preferably not incubated before application to the defect.

These methods may also include the addition of a compound to the cell-matrix combination that improves regeneration of the tissue, as discussed above in the context of the tissue regeneration composition. The compound can be, for example, an antibiotic, an antifibrotic agent, a factor that enhances nerve regeneration, a transcriptional regulator, a platelet derived growth factor, a fibroblast growth factor, an insulin-like growth factor, a bone morphogenic protein (BMP), a parathyroid hormone, a parathyroid hormone-like related protein, a growth hormone, a vascular endothelial growth factor, a transforming growth factor, an Oct4, a Nanog, a Runx2/Cbfal, an Osterix, a Sox9, a DLX2-6, a Msx, a bone sialoprotein, a dentin sialophosphoprotein, a matrix Gla protein, an osteopontin, or a soluble BMP receptor. A preferred example of the compound is a bone morphogenic protein (BMP), most preferably BMP-7. The compound can advantageously also be an antifibrotic agent. Preferred examples include pentifylline (PTF) or pentoxifylline (PTX).

In these methods, preferably the tissue is bone, the vector is a retrovirus, the recombinant SHH gene is operably linked to a β-actin enhancer and promoter, and the mammalian cell is a gingival cell or a periosteum cell. More preferably, the tissue is bone, the vector is a retrovirus, the recombinant SHH gene is operably linked to a β-actin enhancer and promoter, and the mammalian cell is a fat-derived fibroblast-like cell. Even more preferably, the tissue is bone, the vector is an adenovirus, the recombinant SHH gene is operably linked to a β-actin enhancer and promoter, and the mammalian cell is a is a gingival cell or a periosteum cell. In the most preferred embodiments, the tissue is bone, the vector is an adenovirus, the recombinant SHH gene is operably linked to a β-actin enhancer and promoter, and the mammalian cell is a fat-derived fibroblast-like cell.

The cell matrix can further comprises an antifibrotic compound in these methods. Thus, the cell preferably further comprises a recombinant lefty gene such that a recombinant lefty protein can be expressed by the cell.

The methods described and/or claimed herewith can be executed without undue experimentation using standard surgical, molecular biological, and clinical methods. A nonlimiting example of a surgical procedure to correct a defect in a patient is as follows. Donor tissue (preferably autologous) is harvested by standard surgical methods. Harvested tissue is then rapidly (preferably within an hour) processed using simple methods to separate the fibroblast-like cells from adipocytes and other cell types to yield an appropriate amount of partially purified (˜85%) fibroblast-like cells. There is generally no need for extensive purification schemes to obtain 100% pure populations of fibroblast-like cells. The presence of some contaminating adipocytes and other cells types is not problematic. See Example. Meanwhile, fibrotic tissue (if any) is removed from the defect. The fibroblast-like cells are combined with the viral vector. Preferably, the adenoviral vector is combined with the cells for a sufficient time for binding of the adenovirus to the cells (e.g., 30-60 minutes) prior to implant. The adenoviral vector-bound cells are then mixed with the alginate/collagen type I matrix material described above and the cell-matrix combination is then implanted onto the defect. In these procedures, the binding step of adenoviral vector to cells occurs ex vivo without cell expansion while the actual gene transfer occurs in vivo at the wound site.

Various parameters useful for determining the progress of the regeneration are measured as follows. These and other methods for monitoring regeneration progress are routine in the art.

Fibroblast proliferation can be indirectly quantitated by measuring RNA levels of genes associated with fibroblast proliferation such as PDGF-B, FGF-2, or TGF-B1 (Nath, 1998). Because increased RNA levels of these cytokines is associated with fibroblast proliferation but not necessarily indicative of fibrosis, histological and IHC analyses could also be used to quantitatively measure fibroblasts in the defects (a measure of fibroblast proliferation). In addition, histological and IHC analyses can be used to ascertain whether a fibrotic process or regeneration is occurring.

To measure inflammatory mediators at the RNA level, DNA microarrays can be used. Inflammatory proteins can also be assessed in a tissue sample by antibody-mediated assays such as ELISA.

In bone, blastema formation can be characterized using 3D micro CT scanning to measure bone density. Using this technique, on harvested sections of defects, the total 3 dimensional volume of new bone can be measured in a highly quantitative manner.

The invention is additionally directed to methods of regenerating tissue at the site of a tissue defect in a mammal. The methods comprise combining a mammalian cell with a vector, embedding the cell-vector combination in a matrix suitable for applying to a tissue defect, then applying the matrix-cell-vector combination to the tissue defect for a time sufficient to regenerate the tissue. In these methods the cell-vector combination is not expanded in culture before the cell-vector-matrix combination is applied to the tissue defect.

Preferably, the tissue defect is of bone, dermis, nervous tissue, or tendon. Most preferably, the tissue defect is of bone. The preferred matrices comprise alginate and collagen type I; the preferred cell is a human cell. Most preferably, the mammal is a human. It is also preferred if the cell is from the same species as the mammal; most preferably, the cell is from the recipient mammal.

In these methods, the cell is preferably a stem cell, a fat-derived fibroblast-like cell, a gingival cell, or a periosteum cell. More preferably, the vector comprises a recombinant sonic hedgehog (SHH) gene encoding an SHH protein having amino acid sequence that is at least 90% homologous to SEQ ID NO:1 or SEQ ID NO:2. The SHH protein here is preferably a wild-type SHH protein; most preferably a human SHH protein.

The matrix used for these methods can further comprise a compound that improves regeneration of the tissue. Examples of such compounds include an antibiotic, an antifibrotic agent, a factor that enhances nerve regeneration, a transcriptional regulator, a platelet derived growth factor, a fibroblast growth factor, an insulin-like growth factor, a bone morphogenic protein (BMP), a parathyroid hormone, a parathyroid hormone-like related protein, a growth hormone, a vascular endothelial growth factor, a transforming growth factor, an Oct4, a Nanog, a Runx2/Cbfal, an Osterix, a Sox9, a DLX2-6, a Msx, a bone sialoprotein, a dentin sialophosphoprotein, a matrix Gla protein, an osteopontin, and a soluble BMP receptor.

Preferably, the vector is a viral vector, most preferably an adenoviral vector.

In these methods, preferably the tissue is bone, the vector is a retrovirus, the recombinant SHH gene is operably linked to a β-actin enhancer and promoter, and the mammalian cell is a gingival cell or a periosteum cell. More preferably, the tissue is bone, the vector is a retrovirus, the recombinant SHH gene is operably linked to a β-actin enhancer and promoter, and the mammalian cell is a fat-derived fibroblast-like cell. Even more preferably, the tissue is bone, the vector is an adenovirus, the recombinant SHH gene is operably linked to a β-actin enhancer and promoter, and the mammalian cell is a is a gingival cell or a periosteum cell. In the most preferred embodiments, the tissue is bone, the vector is an adenovirus, the recombinant SHH gene is operably linked to a β-actin enhancer and promoter, and the mammalian cell is a fat-derived fibroblast-like cell.

Preferred embodiments of the invention are described in the following example. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims, which follow the example.

Example. Sonic hedgehog gene-enhanced tissue engineering for bone regeneration

These results are published as Edwards et al. (2005).

### EXAMLPE SUMMARY

Improved methods of bone regeneration are needed in the craniofacial rehabilitation of patients with significant bone deficits secondary to tumor resection, congenital deformities, and prior to prosthetic dental reconstruction. In this study, a gene-enhanced tissue-engineering approach was used to assess bone regenerative capacity of Sonic hedgehog (SHH)-transduced gingival fibroblasts, mesenchymal stem cells, and fat-derived cells delivered to rabbit cranial bone defects in an alginate/collagen matrix. Human SHH cDNA isolated from fetal lung tissue was cloned into the replication-incompetent retroviral expression vector LNCX, in which the murine leukemia virus retroviral LTR drives expression of the neomycin-resistance gene. The rat β-actin enhancer/promoter complex was engineered to drive expression of SHH. Reverse transcriptase-polymerase chain reaction analysis demonstrated that the transduced primary rabbit cell populations expressed SHH RNA. SHH protein secretion was confirmed by enzyme-linked immunosorbent assay (ELISA). Alginate/type I collagen constructs containing 2×10⁶ SHH-transduced cells were introduced into male New Zealand White rabbit calvarial defects (8 mm). A total of eight groups (N=6) were examined: unrestored empty defects, matrix alone, matrix plus the three cell populations transduced with both control and SHH expression vectors. The bone regenerative capacity of SHH gene enhanced cells was assessed grossly, radiographically and histologically at 6 and 12 weeks postimplantation. After 6 weeks, new full thickness bone was seen emanating directly from the alginate/collagen matrix in the SHH-transduced groups. Quantitative two-dimensional digital analysis of histological sections confirmed statistically significant (P<0.05) amounts of bone regeneration in all three SHH-enhanced groups compared to controls. Necropsy failed to demonstrate any evidence of treatment-related side effects. This is the first study to demonstrate that SHH delivery to bone defects, in this case through a novel gene-enhanced tissue-engineering approach, results in significant bone regeneration. This encourages further development of the SHH gene-enhanced tissue-engineering approach for bone regeneration.

Introduction

In this study, the SHH gene from human fetal lung tissue was cloned into the replication-incompetent retroviral expression vector series LN (Miller and Rosman, 1989). The rat β-actin enhancer/promoter was engineered to drive expression of SHH. In order to identify a cell population having the best osteogenic potential, three different primary cell populations, gingival fibroblasts, fat-derived stem cells, and periosteal-derived cells, were genetically enhanced with the SHH vector. SHH expression was confirmed in transduced cells at the RNA and protein levels by reverse transcriptase-polymerase chain reaction (RT-PCR) and enzyme-linked immunosorbent assay (ELISA). Cells were introduced into adult (age 6 months, weighing 2.8-3.4 kg) male New Zealand White rabbit calvarial defects using a novel alginate/type I collagen composite bone graft matrix. A total of eight groups (N=6) were examined: unrestored empty defects, matrix alone, matrix plus the three cell populations transduced with both control and SHH expression vectors. The bone regenerative capacity of SHH gene-enhanced cells was assessed grossly, radiographically and histologically at 6 and 12 weeks postimplantation.

SHH induces the expression of multiple BMPs, thus mimicking the complex mixture of BMP heterodimers normally present in developing bone. With the present invention, we expected that the SHH-transduced cells would secrete SHH, resulting in the coordinated downstream expression of multiple bone growth factors implicated in bone repair and regeneration, including the BMPs. Genetic enhancement of cells with SHH should result in better bone repair than methods employing direct protein delivery or genetic enhancement approaches using individual BMPs.

Results

Cloning of human SHH cDNA. We used human fetal lung tissue as the source of total RNA for cDNA cloning of SHH by RT-PCR (Pepicelli et al., 1998), since SHH is highly expressed in developing lung. Owing to the high GC content of the SHH cDNA, it was not possible to isolate an undeleted SHH cDNA as a single contiguous fragment. Instead, the SHH cDNA was assembled using conventional molecular biology techniques from five smaller fragments. In one particularly unstable GC-rich section of the SHH gene, silent mutations were incorporated into the nucleotide sequence to reduce the GC content without altering the amino-acid sequence (see Table 1). With this exception, DNA sequencing of the entire SHH 5′ untranslated region and coding sequence confirmed the reported human SHH sequence (Marigo et al., 1995).

Table 1—Oligonucleotide primers used to generate complete SHH cDNA. Silent mutations (capital letters) were created in the SHH coding sequence of primers NS189 and NS190, which were annealed together to generate a 91 bp fragment. The mutations were needed to reduce the GC content of this region of SHH, which were unstable in the constructs. All other primers were used in RT-PCR to generate the PCR fragment lengths indicated.

| | | | |
|---|---|---|---|
| | | Fragment | SEQ ID |
| Name | Sequence | Size | NO |

| | | | |
|---|---|---|---|
| NS145 F | aaaaagcttgggcgagatgctgctgctggcgagatgtct | 404 bp | 3 |
| NS182 R | tcgtcccagccctcggtcacccgc | | 4 |
| NS181 F | gcgggtgaccgagggctgggacga | 359 bp | 5 |
| NS115 R | aggaaagtgaggaagtcg | | 6 |
| NS198 F | accgcgtgctggcggcggacgaccaggg | 520 bp | 7 |
| NS199 R | tgtgcgcgcgggcgccagtgcagccaggagcgcg | | 8 |
| NS189 F | cccgcgcgcacAgaTAgAggAggAgaTag | | 9 |
| | TggTggAggTgaTAgAggAggTggTgg | | |
| | AggAagagtagccctaaccgctccaggtgctgccg | | |
| NS190 R | cggcagcacctggagcggttagggctactctTccTcc | | 10 |
| | AccAccTccTcTAtcAccTccAccActAtcTcc | | |
| | TccTcTAtcTgtgcgcgcggg | | |
| NS200 F | ctccaggtgctgccgacgctccgggtgcgg | 148 bp | 11 |
| NS146 R | tttatcgattcagctggacttgaccgccatgcccagcgg | | 12 |

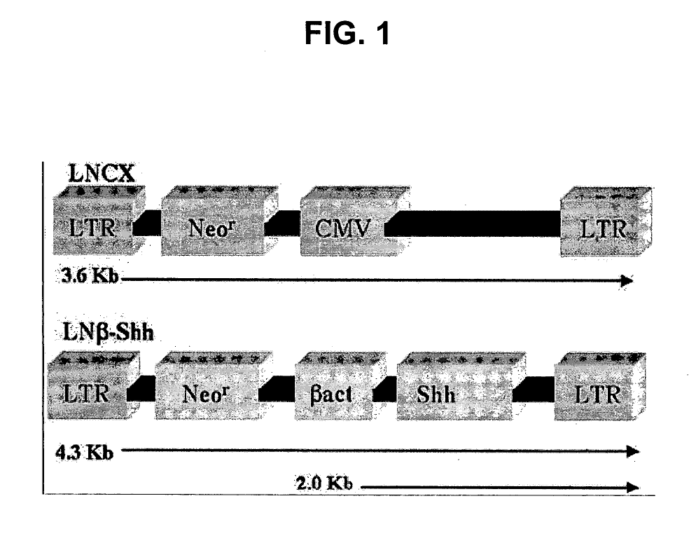

Retroviral vector plasmid and particle generation. SHH cDNA was cloned into the retroviral expression vector LNCX, based on the LN series of vectors in which the murine leukemia virus retroviral LTR drives expression of the neomycin-resistance gene (Miller and Rosman, 1987). In the retroviral vector plasmid pLNB-SHH, the SHH cDNA was cloned as a HindIII/ClaI fragment replacing the BMP-7 HindIII/ClaI fragment in plasmid pLNB-BMP-7 (FIG. 1). The β-actin enhancer/promoter was chosen for driving expression of SHH because it is a weaker ‘housekeeping’ enhancer/promoter than the strong viral cytomegalovirus (CMV) enhancer/promoter, which can result in harmful overexpression of potent cytokines and morphogens (Mason et al., 1998). We had previously noted that overexpression of BMP-7 under control of the CMV promoter was toxic to our peristeal-derived cells (Mason et al., 1998). Thus, we performed limited dosing experiments by testing strong and weak enhancer/promoters to drive transgenes in primary mesenchymal stem cells and other cells in vitro. We found that the β-actin enhancer/promoter is preferred due to its reasonable expression levels with resultant lack of toxicity.

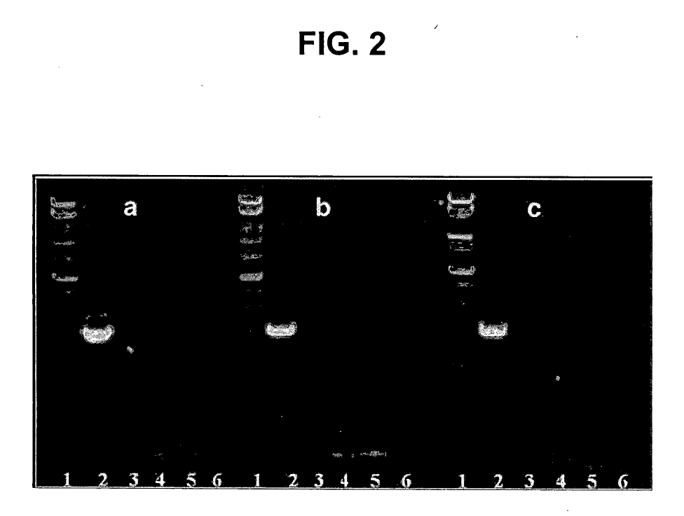

RT-PCR analysis of SHH expression in the selected primary cell populations. Variations in the signaling range of SHH appear to be due to tissue-specific differences in intracellular processing and tissue-restricted expression of binding proteins. Consequently, three cell types, all of which are in plentiful supply and easily harvested, were analyzed: gingival fibroblasts, fat-derived stem cells, and periosteal-derived cells. SHH RNA expression was confirmed in the three transduced cell lines by RT-PCR using vector-specific primers. Our results (FIG. 2) showed that the LNB-SHH-transduced periosteal cells expressed SHH at the RNA level, while control-transduced cells did not. The gingival fibroblasts and fat-derived cells gave similar results. Controls included GAPDH for RNA integrity and reverse transcription positive controls, and no template as negative control.

SHH protein production in transduced cells as assessed by ELISA. Cells were grown in T-75 flasks to confluence at a concentration of 1-4×10⁶ total cells. A 48 h low serum conditioned media was harvested from the three cell lines (gingival fibroblasts, periosteal, and fat-derived stem cells) carrying the LNCX or LNB-SHH constructs. Indirect ELISAs were performed using a goat IgG anti-mouse SHH amino-terminal peptide primary antibody, followed by color development using a biotinylated secondary antibody (mouse anti-goat IgG biotinylated antibody and horseradish peroxidase conjugate). Background levels in the control-transduced cells were subtracted out to arrive at the final value.

Our results confirmed that the three transduced cell lines were expressing and secreting SHH at the protein level in vitro. SHH-transduced periosteal stem cells secreted the greatest amount of SHH. SHH4-transduced cells secreted the following:

LNB-SHH-transduced periosteal-derived cells, 19 ng SHH/10⁶ cells/24 h; LNB-SHH-transduced fat-derived stem cells, 5 ng SHH/10⁶ cells/24 h; LNB-SHH-transduced gingival fibroblasts, 3 ng SHH/10⁶ cells/24 h.

This is comparable to the level of BMP production previously observed in osteochondral defect studies using LNB-BMP-7-transduced periosteal-derived cells Grande et al., 2001.

Assembly of alginate/type I collagen/cell composites and assessment of in vitro viability. The matrix is a critical component of any tissue-engineering protocol involving anchorage-dependent cells. Several different materials and various combinations of materials were assessed for use as a matrix prior to selection of the alginate/type I collagen mixture. Matrigel (BD Biosciences, Franklin Lakes, N.J., USA), gelatin, agar, Gelfoam (Johnson and Johnson, Summerville, N.J., USA), and BioOss (Luitpold Pharmaceuticals, Shirley, N.Y., USA) all gave inferior handling and cell compatibility properties. Consequently, we developed a novel alginate/type I bovine collagen-based matrix in which the alginate provides a structural mesh around the cells and the collagen supplies the desired osteoconductive properties to the graft.

Alginate is a biodegradable polysaccharide composed of mannuronic and guluronic acid units. The porous nature of alginate gels allows for the migration of cells and regulatory proteins inside the network (Stabler et al.; 2001). The alginate used in these studies was from Macrocystitis pyrifera (kelp) with a medium viscosity and is composed of 61% mannuronic and 39% guluronic acid and a molecular weight of 100,000 Da. Type I collagen (1%) was added to increase the osteoconductive potential of the alginate (Fleming et al., 2000).

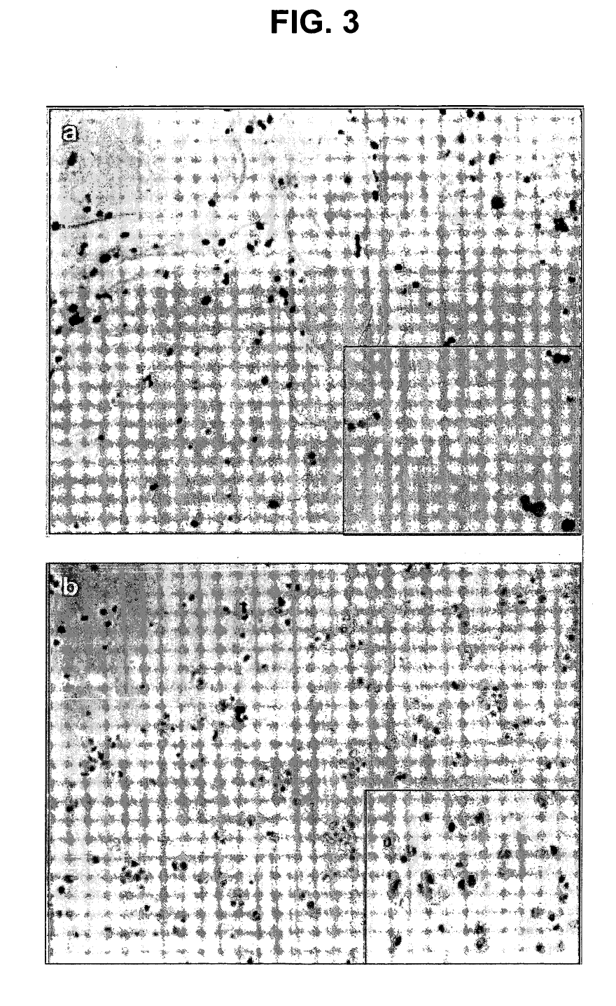

Although our experimental protocol did not require culturing of transfected cells in the graft material prior to implant into the defects, it was necessary to first assess the viability of cells in this composite bone graft material. Consequently, composites of alginate/type I collagen/periosteal-derived cells were prepared and submitted for histological sectioning immediately after assembly (time 0) and after 7 days in culture. It was noted that the cells were evenly distributed throughout the graft material at time 0 (FIG. 3a). After 1 week in culture, the cells were healthy and had expanded in clusters throughout the graft (FIG. 3b), demonstrating the suitability of this graft material.

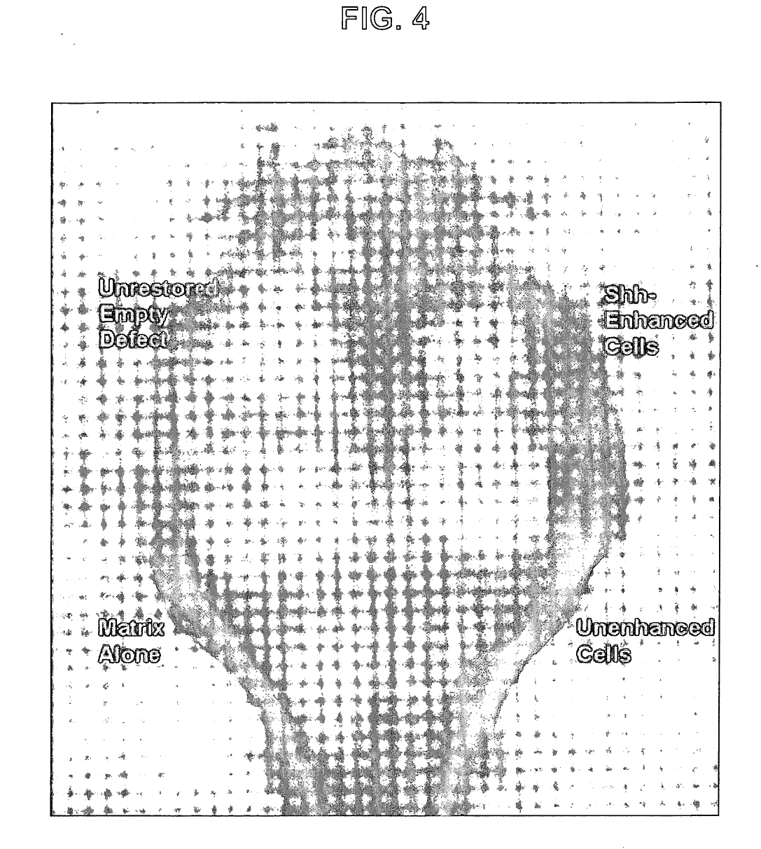

Bone regeneration at 6 and 12 weeks. Adult male (age 6 months) New Zealand White rabbits were used for the in vivo assessment of bone regeneration. Full thickness 8 mm cranial bone defects (frontal and parietal bones, four defects per rabbit) were created using a trephine bur. The surgically created defects were restored with the selected transduced cells or the corresponding controls in the alginate/type I collagen matrix. Dosing was accomplished at the cell number level, not at the promoter level. In all, 2×10⁶ cells were implanted per defect. The experimental groups comprised allogenic gingival fibroblasts, periosteal and fat-derived stem cells transduced with the replication-incompetent SHH retroviral vector (LNB-SHH) and control vector (LNCX). Additional controls included alginate/collagen matrix alone and empty defects. A total of 12 calvarial defects (six per time/group) were analyzed for each experimental group. After 6 or 12 weeks, the animals were killed and post-mortem radiographs were taken. FIG. 4 is representative of the radiographic results seen at 6 weeks for all cell types. Empty defects, matrix alone, and control-transduced cells show minimal levels of bone regeneration. Conversely, SHH-transduced cells show very substantial levels of bone regeneration radiographically.

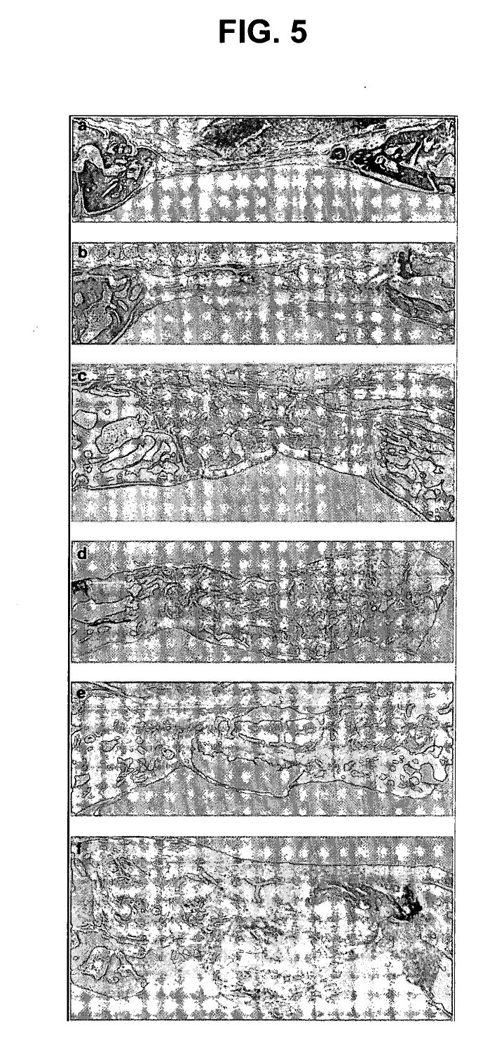

The defect sites were identified visually and submitted for histologic examination. Composite photomicrographs were assembled from histological sections that were taken through the center of the defects. Only a thin layer of fibrous connective tissue formed in the unrestored empty defect group (FIG. 5a). Matrix alone (without cells) integrated into the defects, but again there was only minimal bone formation (FIG. 5b). However, in all matrix-containing groups, the thickness of the defect space was effectively preserved. The defects restored with control-transduced cells plus matrix demonstrated only minimal bone formation (FIG. 5c). At higher magnification, control-transduced cells within the matrix could still be seen after 6 weeks in vivo.

Conversely, SHH gene enhancement of the periosteal-derived cells resulted in the formation of new bone, primarily along the edges of the defect (FIG. 5d). This new bone was composed of thin trabeculae, and had a somewhat delicate appearance. The SHH-enhanced fat-derived stem cells appeared to form relatively thick trabeculae of bone, but this new bone was not always well dispersed throughout the defect (FIG. 5e). Even dispersal of new bone was complicated by the observation that the use of fat-derived stem cells often resulted in growth of cyst-like structures in both the control-transduced and gene-enhanced groups. These cyst-like structures were not seen with periosteal-derived cells or gingival fibroblasts.

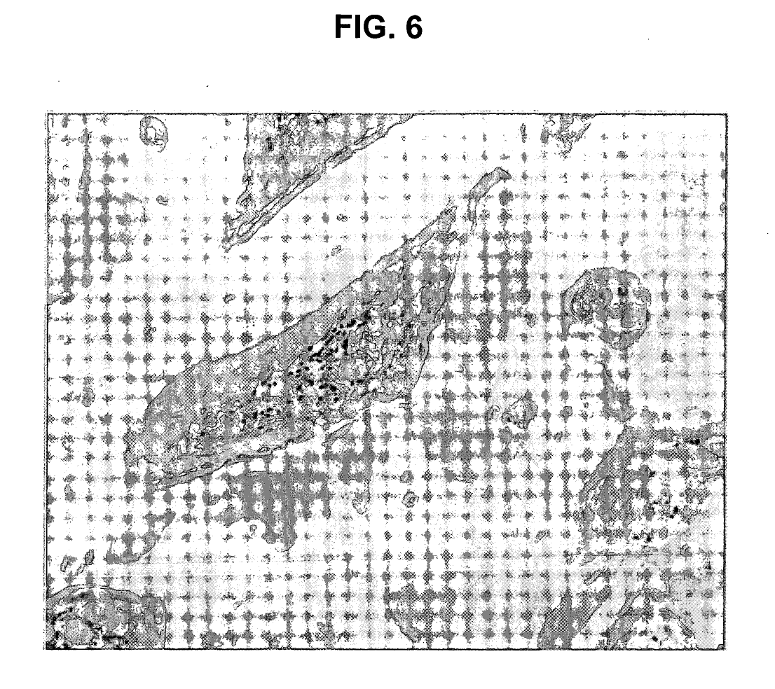

The best-dispersed bone was seen with the SHH-transduced gingival fibroblasts, where a substantial amount of new bone formation was noted throughout the matrix (FIG. 5f). Equally significant was the thickness of this new bone. On high-power histologic examination, the new bone was shown to be emanating directly from the matrix (FIG. 6).

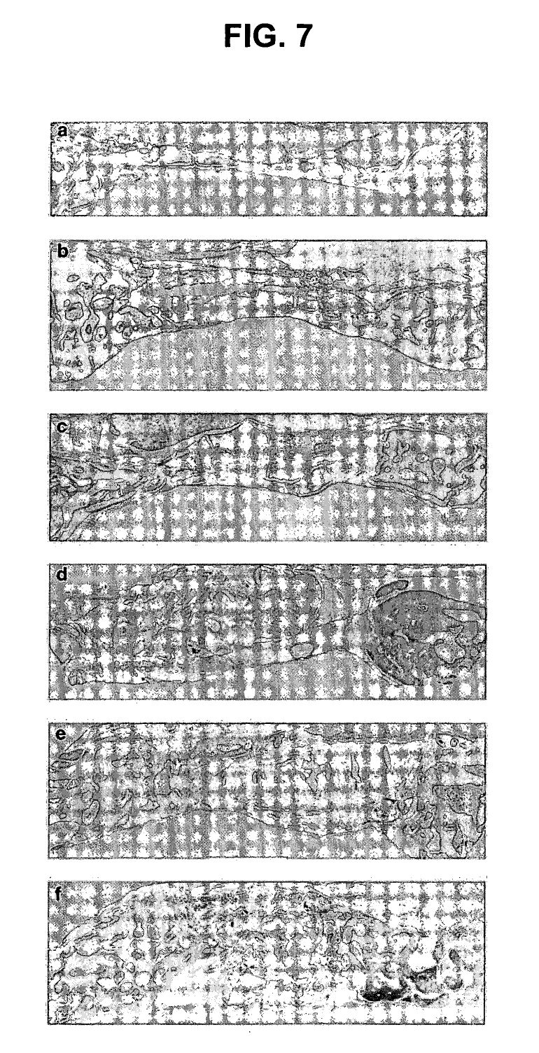

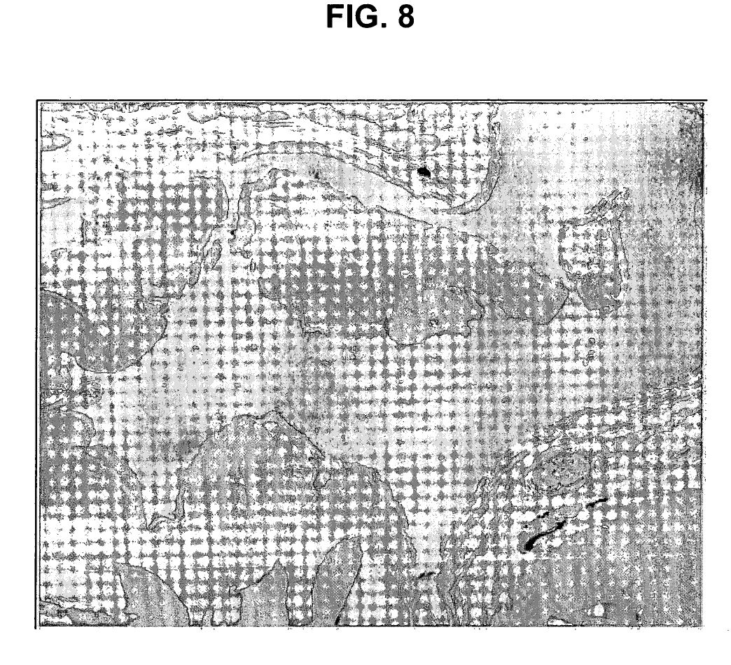

Results at 12 weeks (FIG. 7a-f) were similar to the findings at 6 weeks. Consistent with the 6-week data, the best-dispersed bone at 12 weeks was seen with the SHH-enhanced gingival fibroblasts, where near full thickness bone formation was observed throughout the defect. At higher magnification, significant new bone formation and bone marrow was evident (FIG. 8). In areas where new bone formation was not complete, the remaining matrix had a lower density of cells, indicating that inclusion of more cells in the implants may prove beneficial.

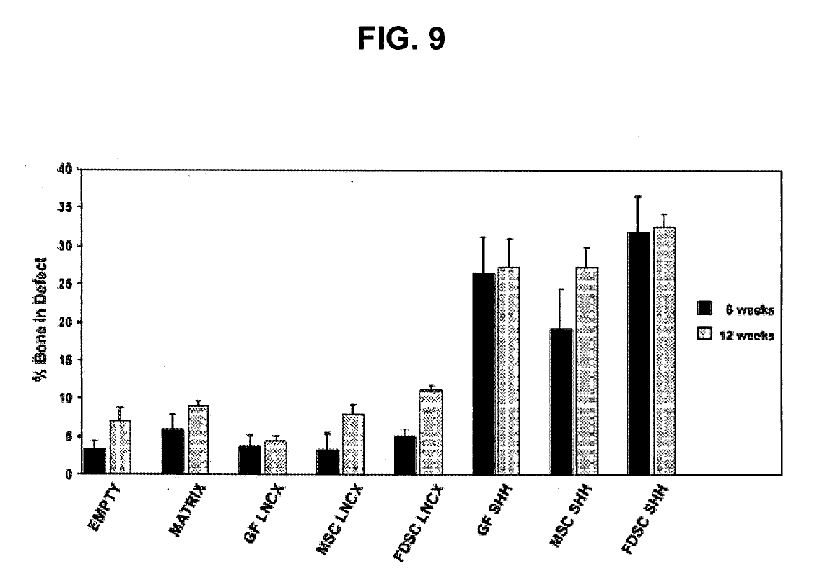

Quantitative digital analysis of histological sections was performed and the total two-dimensional amount of new bone was determined. Briefly, digitized composite photomicrographs were analyzed on an IBM PC running Windows 98 with Adobe Photoshop 6.0. The mineralized area of the defects in the digitized radiographs was identified by the value of the pixel in the image. The percentage of area of mineralized tissue within the defect size was determined. One-way analysis of variance (ANOVA) was followed by pairwise comparison. A comparison of bone regeneration at 6 and 12 weeks showed statistically significant new bone formation in all three SHH-enhanced cell lines at both time points compared to controls (FIG. 9).

Finally, regarding the safety of stably transducing cells to express SHH in vivo, autopsies performed on SHH-transduced rabbits failed to demonstrate any evidence of treatment-related side effects after 12 weeks.

Discussion

The adult rabbit calvarial ‘critical size defect’ model was chosen because the cranial bones, like the maxilla and mandible, are formed through intramembranous ossification (Rudert, 2002; Hollinger and Kleinschmidt, 1990). This model has been extensively investigated and characterized with regards to its intrinsic bone healing capacity (Frame, 1980). While it has been reported (Tsuchida et al., 2003) that allogeneic cell-mediated femoral bone regeneration in the rat model requires immune suppression, we have not observed tissue rejection-related problems using a rabbit calvarial defect model. It may be that the allogenic cells from different New Zealand White rabbits are not as antigenically heterogenous as the cells from the two different rat strains employed by Lou and co-workers (Tsuchida et al., 2003). Alternatively, the rabbit calvarial site might be immune privileged.

Based on the early work of Frame (1980), a critical size calvarial defect (CSD; defined as a defect that will not heal completely during the lifespan of the animal) in the adult rabbit was determined to be 15 mm in diameter, when examined 24 weeks postsurgery. However, the concept of CSD is in flux. Since most studies are of limited duration and do not extend over the life of the animal, the CSD is now being redefined as the size of the defect that does not heal over the length of the study (Gosian, 2000). Previous definitions of CSD were based on a two-dimensional, linear measurement of bone formation, and did not take into account the overall thickness of the new bone. Consequently, cranial defects that developed a continuous, even if very thin, shelf of bone over the surgical site were considered healed. However, the most important parameter of success in bone healing is the total three-dimensional amount of new bone deposited in the defect, because the goal in most craniofacial applications of bone regeneration is to restore the site to its original three-dimensional state. Therefore, an 8 mm defect size was chosen since there is ample evidence (Kramer et al., 1968) to suggest that this sized defect does not heal spontaneously over a 12-week period.

Our results clearly demonstrate that minimal bone regeneration occurred in empty 8 mm defects, which validates this size defect for study of bone regeneration at time points up to 12 weeks.

Control-transduced cells were used as the best control group for these studies. Control-transduced cells were genetically enhanced with the neomycin-resistance gene and selected in G418. SHH-transduced cells were treated identically as control cells, with the exception that the vector they were transduced with contained the SHH gene driven from the β-actin promoter in addition to the neomycin-resistance gene. We chose not to use nontransduced cells as a control because in previous experiments in which nontransduced cells were used, there was no statistical difference in bone regeneration between the control-transduced and nontransduced groups (Breitbart et al., 1999). Consequently, the control-transduced cells serve as the most suitable control for these studies.

The matrix is a critical component of any tissue-engineering protocol involving anchorage-dependent cells. Ideally, the matrix should be easy to handle, allow for adherence of cells, and provide a three-dimensional scaffold of sufficient strength to hold the defect space. It must also be porous enough to allow for the free diffusion of cells and growth factors. Purified bovine collagen is biocompatible, and because it promotes the mineralization process, it is also osteoconductive. However, we found that a collagen-based system alone did not afford a matrix with the requisite strength to hold the defect space. Moreover, collagen gels tend to contract and lose their shape and consistency after as little as 12 h in culture (Diduch et al., 2000).

Alginate hydrogels are used extensively in cell encapsulation and tissue-engineering applications because of their structural properties and good biocompatibility (Milla et al., 2001; Loebsack et al., 2001). The porous nature of alginate gels allows for the migration of cells and cytokines inside the network. Bone marrow stromal cells embedded in alginate alone have been used to regenerate rabbit osteochondral defects (Diduch et al., 2000) and sheep cranial defects (Shang et al., 2001), with no evidence of a host immune response. While alginate gels alone support cell proliferation, proliferation can be enhanced by the addition of an osteoconductive material to the matrix (Miralles et al., 2001).

Our results demonstrate improved bone regeneration through the use of a novel alginate/type I bovine collagen-based matrix in which the alginate provides a structural mesh around the cells and the collagen supplies the desired osteoconductive properties to the graft.

Variations in the signaling range of SHH appear to be due to tissue-specific differences in intracellular processing and tissue-restricted expression of binding proteins. This suggests that the ability of cells to respond to SHH may be dependent on the stage of differentiation of the particular cell, with only immature pluripotential cells being capable of differentiating into an osteoblastic lineage (Spinella-Jaegle et al., 2001). Consequently, three cell types originating from different tissues were analyzed: gingival fibroblasts, fat-derived stem cells and periosteal-derived cells. All of these cell types are in plentiful supply and easily harvested. Gingival fibroblasts can be induced to express an osteoblastic phenotype (Murphy et al., 2001; Krebsbach et al., 2000). Tissue obtained by liposuction contains a fibroblast cell-like population (fat-derived stem cells) that can be induced to differentiate into bone when placed in an appropriate medium (Zuk et al., 2001). Periosteal-derived cells were selected because of their proven ability to repair bone defects when transfected with BMP-expressing retroviral vectors (Mason et al., 1998).

In this study, our objective was to determine if SHH genetic enhancement using the identical vector system would improve bone regenerative capacity of the chosen cell types over control-transduced cells. Therefore, no attempt was made to control the level of SHH expressed from the different cell types, as promoters operate at unpredictable levels in different cell types. Dosing was accomplished at the cell number level, not at the promoter level.

The selection of the number of cells implanted per defect (2×10⁶ cells) was based on the dose-response curves of Gysin et al (2002), who demonstrated that the optimum cell count for an 8 mm calvarial defect was 1-2×10⁶ BMP-expressing cells. Additionally, our in vitro experiments (FIG. 3a and b) established that a density of 2×10⁶ cells/450 μl of construct afforded an acceptable cell density at the histologic level. However, our in vivo results suggest that this total cell count may be insufficient for complete bone regeneration at 12 weeks. In some areas where new bone formation was not complete, the remaining matrix had a lower density of cells. Increasing the concentration of cells should result in faster and more complete bone regeneration.

The use of gene-enhanced tissue engineering may overcome limitations associated with the one-time delivery of a bolus of protein by providing a sustained, local delivery of protein factors. In this study, a replication-incompetent retroviral expression vector based on the LN series (Miller and Rosman, 1989) was used. In this vector, the relatively weak rat β-actin enhancer/promoter was used to drive expression of SHH. Overexpression of potent morphogens under control of the stronger CMV enhancer/promoter or from other transient expression systems that grossly overexpress transgenes can be toxic (Mason et al., 1998). The retroviral vectors used in this study were engineered for sustained local delivery of physiologic levels of the expressed gene. In prior studies (Mason et al., 1998), we reported that expression of BMP-7 in periosteal-derived cells transduced with similar retroviral vectors was measurable for several weeks prior to loss of expression.

Other systems could have been used which result in local presence of supraphysiologic levels of protein for relatively short periods of time, but this would not have mimicked what occurs during the normal course of early skeletogenesis; a process we are trying to emulate. Although a retroviral vector system was used in this study, other gene delivery systems with greater clinical applicability for bone regeneration that do not require ex vivo cell culture and that result in sustained presence of physiological levels of transgene expression could also be used in future studies.

We demonstrated that SHH delivery to bone defects, in this case through a novel gene-enhanced tissue-engineering approach, resulted in significant bone regeneration. It is interesting to note that all three cell types, selected for use in these studies because of their reported bone regenerative capacity, were capable of regenerating bone but only when genetically enhanced with SHH. In addition, although the SHH gene-enhanced fat-derived stem cells proved capable of regenerating bone, the unexpected formation of cyst-like structures observed with the use of these cells requires further study to determine impact on long-term bone regeneration.

Our ELISA data support the hypothesis that only low levels of transgene expression are needed to heal cranial defects; the level of expression being only one component of a cell's ability to stimulate bone regeneration. It is our contention, and that of others (Kato et al., 2001; Goetz et al., 2002) that a gradient of secreted morphogen is needed for optimal bone regeneration. There are different ways to try to attain such a gradient in vivo. The retroviral system employed in this study allows for the secretion of modest amounts of morphogen over an extended period of time, which results in the build up of efficacious morphogen levels locally.

It was not determined in this study whether the transduced cells are differentiating into osteoblasts or instead are triggering other uncommitted mesenchymal cells that have migrated into the defect site to differentiate into osteoblasts, although the former is suspected. In the future, in situ hybridization experiments will be performed to determine whether the bone cells in the regenerated grafts contain the vector, indicating transdifferentiation into bone as found in our earlier osteochondrat tissue regeneration studies (Breitbart et al., 1999).

In conclusion, this is the first study to demonstrate that SHH delivery to bone defects, in this case through a novel gene-enhanced tissue-engineering approach, results in significant bone regeneration. This encourages further development of the SHH-mediated tissue-engineering approach for bone regeneration.

Materials and Methods

Approval of experimental protocols. The protocol was approved by the North Shore-Long Island Jewish Health System Institutional Biosafety Committee. Animal protocols were approved by the North Shore-Long Island Jewish Health System Institutional Animal Care and Use Committee.

Isolation and culture of primary cell populations. Rabbit periosteal-derived cells. Rabbit periosteum was harvested from the anteromedial surface of the proximal tibia of male New Zealand White rabbits. A rectangular incision was made to expose the bone and periosteum was separated from underlying bone. Only the cambium layer was harvested (confirmed by histological observation). Harvested periosteum was diced into 1 mm cubes and cultured in SDMEM media (composed of high glucose DMEM supplemented with 10% heat-inactivated fetal bovine serum, 1× antibiotic/antimycotic, 12 mM HEPES, 0.4 mM L-proline, and 50 mg/L ascorbic acid).

Fat-derived stem cells. Fat tissue was harvested from the inguinal and abdominal regions of male New Zealand White rabbits. The tissue was placed in SDMEM and digested with 0.075% collagenase/DNAse mixture at 37° C. in a 5% CO₂ incubator for 1 h. The cell suspension was then filtered through a 100 nm NYTEC filter, the cells centrifuged, washed twice, and cultured in SDMEM.

Gingival fibroblasts. Gingival tissue was harvested from the palate of male New Zealand White rabbits. The tissue was cut into 1 mm explants and cultured in SDMEM at 37° C. in humidified 5% CO₂.

Construction of retroviral expression vectors. The SHH cDNA, isolated from human fetal lung tissue, had previously been cloned into the retroviral expression vector LNCX, based on the LN series of vectors in which the murine leukemia virus retroviral LTR drives expression of the neomycin-resistance gene (Miller and Rosman, 1989). In the retroviral vector plasmid pLNB-SHH, the SHH cDNA was cloned as a HindIII/Clal fragment replacing the BMP-7 HindIII/Clal fragment in plasmid pLNB-BMP-7. The rat β-actin enhancer/promoter, a relatively weak housekeeping promoter with low-level constitutive expression, was chosen to drive expression of SHH because expression of potent morphogens from this promoter is not toxic to cells. The retroviral vector plasmids were CaPO₄ transfected into GP+E 86 cells (Markowitz et al., 1988). Retroviral vector particle containing conditioned media was collected 48 h post-transfection and used to transduce PA317 cells in the presence of 8 g/ml polybrene (Miller and Buttimore, 1986). PA317 cells were selected for 10-12 days in D10 medium supplemented with 300 μ/ml active neomycin analog G418. Amphotropic retroviral vector particles were collected from a cloned producer cell line having a titer of 1×10⁶ Neo CFU/ml.

Transduction and selection of cells. Cells were transduced at 25% confluence in six-well dishes using 400 μl of retroviral vector particles and 1.6 ml D10 supplemented with 8 μ/ml polybrene. Two separate transductions were performed on consecutive days. Kill control experiments determined that the 10 day selective conditions for rabbit periosteal-derived cells, fat-derived stem cells, and gingival fibroblasts are 600, 1800, and 900 μg/ml active G418, respectively. Populations of resultant G418 selected rabbit cells were used in all studies.

RT-PCR analysis of SHH expression. Total RNA was isolated from 1×10⁶ transduced cells using the RNeasy kit (Qiagen). First strand synthesis was performed using the Reverse Transcription System (Promega). SHH RT-PCR was performed using Herculase Hot Start Enhanced DNA Polymerase (Stratagene) as follows: annealing temperature 60° C.; 30 s to anneal; 72° C. extension temperature; 2 min to extend; 30 cycles. Oligonucleotide PCR primers NS145 5′ aaaaagcttgggcgagatgctgctgctggcgagatgtct 3′ (SEQ ID NO:3)(forward primer in 5′ coding sequence of SHH) and NS239 5′ ccctttttctggagactaaataaaatc 3′ (SEQ ID NO:13)(reverse primer downstream of the SHH gene in the viral vector) were used to amplify a 1446 bp vector-specific SHH transcript encompassing the 5′ end of human SHH and flanking vector sequence encoded specifically by LNB-SHH.

GAPDH primers NS159 (5′ ggtcatccctgagctgaacg 3—SEQ ID NO:14) and NS160 (5′ ttcgttgtcataccaggaaat 3—SEQ ID NO:15), at an annealing temperature of 55° C. (30 s to anneal; 72° C. extension temperature, 1 min to extend; 30 cycles), were used to generate an expected 294 bp GAPDH transcript as control of RNA quality. A no template control was also included.

ELISA of SHH secretion by transduced cells. Cells were grown to confluence in SDMEM supplemented with G418 at a concentration of 1-4×10⁶ total cells. A 48-72-h conditioned, low serum media (Optimem; Gibco) was harvested from the three cell lines (gingival fibroblasts, periosteal and fat-derived stem cells) carrying the LNCX or LNB-SHH constructs. Indirect ELISAs were performed by adding 100 μl of conditioned media into 96-well flat-bottom Maxisorp plates (Nunc, Roskilido, Denmark). All assays were performed in triplicate. Antigen was bound at 37° C. for 1 h, blocked with 200 μl PBS-T (Phosphate Buffered Saline with 0.1% Tween-20) for 1 h at room temperature, and then washed three times with PBS-T. The primary antibody, goat IgG anti-mouse SHH amino-terminal peptide (100 μg/ml; R&D Systems; Minneapolis, Minn., USA), was diluted 1:100 in PBS-T, and 100 μl was added per well for 2 h at room temperature. This antibody cross reacts with human SHH. Three washes with PBS-T were followed by development using a biotinylated secondary antibody (mouse anti-goat IgG biotinylated antibody; Vectastain ABC kit, Vector Laboratories; Burlingame, Calif., USA) and horseradish peroxidase conjugate (Vectastain ABC kit). The chromogenic substrate tetramethylbenzidine (TMB Microwell Peroxidase Substrate; KPL, Gaithersburg, Md., USA) was used for color development. The plates were read at OD450 using a model 400 ATC ELISA plate reader (SLT Lab Instruments; Grodig, Austria). Unconditioned Optimem was used as background control. The background reactivity present in the control-transduced cell lines was subtracted from the raw values to arrive at a final determination of SHH protein production in the transduced cell lines (ng SHH/1×10⁶ total cells/24 h).

Assembly of gene-modified cell—alginate-collagen matrix constructs. A solution of purified type 1 bovine collagen (Vitrogen 100, 3.1 mg/ml collagen; Cohesion, Palo Alto, Calif., USA) was prepared by adding 800 μl of Vitrogen 100 to 100 μl of 10× PBS, followed by the addition of 100 μl of 0.1 M NaOH.

The gene-modified cell lines were trypsinized and the cell pellets, each containing 2.0×10⁶ cells, were resuspended in a 50 ml Falcon tube (Becton Dickinson; Lincoln Park, N.J., USA) in 200 μl of 2.0% alginic acid (sodium salt, medium viscosity, from Macrocystis pyrifera; Sigma, St Louis, Mo., USA). The cell-alginate solution was added to 200 μl of the above-prepared type 1 collagen preparation. Initial gelation was accomplished by placing the cell-alginate-collagen amalgam at 37° C. for 30 min. Gelation of the alginate was completed by adding 4 ml of 100 mM CaCl₂ directly to the amalgam. The matrix was allowed to gel for 15 min and then rinsed three times with PBS prior to implantation.

Surgical procedures. A total of 24 adult male (age 6 months) New Zealand White rabbits, weighing 3.0-4.0 kg, were used in this study. The rabbits were kept in standard laboratory double cages with a 12-h day/night cycle and an ambient temperature of 21° C. The rabbits were permitted 2 h free housing per day, and had access to tap water and food pellets.

Food and water were withheld from the rabbits for 6 and 1 h, respectively prior to surgery. A total of 0.4 cc/3 kg of Tazidine was administered 18 h prior to surgery by means of intramuscular injection. Animals were preanaesthetised with an intramuscular injection of 5 mg/kg acepromazine, and induced with 12.5 mg/kg ketamine and 4% isoflurane. Adequateness of anaesthesia was assessed by the absence of withdrawal reflex to toe pinch and the absence of corneal reflex.

In each animal, the surgical field was shaved and prepped with iodophor. Following the infiltration of local anaesthesia (2% lidocaine with 1:100 000 epinephrine), midline sagittal incisions were extended from the occipital region to the bridge of the nose. Subperiosteal dissections were performed anteriorly and posteriorly to expose the frontal and parietal regions of the cranium. Using a trephine bur with saline irrigation, four full thickness 8 mm bone defects were created. The surgically created defects were restored with the selected transduced cells in the alginate matrix or the corresponding controls. The scalp tissues were reapproximated to the remaining calvarium, and sutured with 4-0 Vicryl sutures. Postoperative analgesia was accomplished by administering 0.1 mg/kg Buprenex subcutaneously q12 h for the first 48 h.

After 6 or 12 weeks, the animals were anesthetized with ketamine and killed by means of a pentobarbital overdose.

Experimental groups. The experimental groups comprised allogenic gingival fibroblasts, periosteal and fat-derived stem cells transduced with the replication-incompetent SHH retroviral vector (LNB-SHH) and control vector (LNCX). Additional controls included alginate/collagen matrix alone and empty defects, for a total of eight groups. Four full thickness 8 mm bone defects were created per animal. The different groups were evenly distributed between the animals and between anterior vs posterior calvarial sites. In total, 12 calvarial defects (six per time/group) were analyzed for each experimental group.

Radiographic analysis of bone defect healing. Post-mortem radiographs (Kodak Ultraspeed DF-50 Dental Film) were taken of the sectioned calvaria using a portable X-ray unit (Philips Dens-o-Matic, 65 kVp, 7.5 mAmp, 1.5 s).

Histological analysis. The defect sites were identified visually, and then sectioned into halves. One half was decalcified in ‘overnight bone decalcification’ solution (Decal Corporation, Tallman, N.Y., USA) for 3 days. After embedding in paraffin, 4 m sections closest to the center of the defect, showing the full diameter of the defects, were obtained and stained with hematoxylin and eosin. When processing the alginate/collagen/cell matrices for histologic examination, 10 mm CaCl₂ was added to the formalin during the initial fixation period to prevent depolymerization of the alginate matrix. The matrices were then fixed overnight with 50 mm BaCl₂ at 4° C. to permanently crosslink the alginate prior to final processing.

Quantitative digital analysis of histological sections was performed. Digitized composite photomicrographs were analyzed on an IBM PC running Windows 98 with Adobe Photoshop 6.0. The mineralized area of the defects in the digitized radiographs was identified by the value of the pixel in the image. The percentage of area of mineralized tissue within the defect size was determined.

Statistical analysis. Determination of n (the minimum sample size to provide proper discriminatory capability) for in vivo animal studies was carried out by power analysis. A sample size of six would yield 80% power to detect a difference of 10% between the two groups (case vs control) using a two-tailed, paired t-test with a 0.05 significance level. ANOVA was followed by pairwise comparison. A ‘P’-value of less than 0.05 was considered statistically significant.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

SEQ ID NOs

SEO ID NO:1—human sonic hedgehog precursor amino acid sequence—from GenBank Q15465

1 mlllarcll vlvssllvcs glacgpgrgf gkrrhpkklt playkqfipn vaektlgasg
61 ryegkisrns erfkeltpny npdiifkdee ntgadrlmtq rckdklnala isvmnqwpgv
121 klrvtegwde dghhseeslh yegravditt sdrdrskygm larlaveagf dwvyyeskah
181 ihcsvkaens vaaksggcfp gsatvhleqg gtklvkdlsp gdrvlaaddq grllysdflt
241 fldrddgakk vfyvietrep rerllltaah llfvaphnds atgepeassg sgppsggalg
301 pralfasrvr pgqrvyvvae rdgdrrllpa avhsvtlsee aagayaplta qgtilinrvl
361 ascyavieeh swahrafapf rlahallaal apartdrggd sgggdrgggg grvaltapga
421 adapgagata gihwysqlly qigtwlldse alhplgmavk ss
   SEQ ID NO:2—rat sonic hedgehog precursor amino acid sequence—from GenBank Q63673
1 mllllarcfl valassllvc pglacgpgrg fgkrqhpkkl tplaykqfip nvaektlgas
61 gryegkitrn serfkeltpn ynpdiifkde entgadrlmt qrckdklnal aisvmnqwpg
121 vklrvtegwd edghhseesl hyegravdit tsdrdrskyg mlarlaveag fdwvyyeska
181 rihcsvkaen svaaksdgcf pgsatvhleq ggtklvkdls pgdrvlaadd qgrllysdfl
241 tfldrdegak kvfyvietre prerllltaa hllfvaphnd sgptpgpspl fasrvrpgqr
301 vyvvaerggd rrllpaavhs vtlreeaaga yapItadgti linrvlascy avieehswah
361 rafapfrlah allaalapar tdgggggsip apqsvaearg agppagihwy sqllyhigtw

421 lldsetlhpl gmavkss

SEQ ID NO:3 - sonic hedgehog oligonucleotide
primer NS145 F
aaaaagcttgggcgagatgctgctgctggcgagatgtct
SEQ ID NO:4 - sonic hedgehog oligonucleotide
primer NS182 R
tcgtcccagccctcggtcacccgc
SEQ ID NO:5 - sonic hedgehog oligonucleotide
primer NS181 F
gcgggtgaccgagggctgggacga
SEQ ID NO:6 - sonic hedgehog oligonucleotide
primer NS115 R
aggaaagtgaggaagtcg
SEQ ID NO:7 - sonic hedgehog oligonucleotide
primer NS198 F
accgcgtgctggcggcggacgaccaggg
SEQ ID NO:8 - sonic hedgehog oligonucleotide
primer NS199 R
tgtgcgcgcgggcgccagtgcagccaggagcgcg
SEQ ID NO:9 - sonic hedgehog oligonucleotide
primer NS189 F (mutations are capitalized)
cccgcgcgcacAgaTAgAggAggAgaTagTggTggAggTgaTAgAggAgg
TggTggAggAagagtagccctaaccgctccaggtgctgccg
SEQ ID NO:10 - sonic hedgehog oligonucleotide
primer NS190 R (mutations are capitalized)
cggcagcacctggagcggttagggctactctTccTccAccAccTccTcTA
tcAccTccAccActAtcTccTccTcTAtcTgtgcgcgcggg
SEQ ID NO:11 - sonic hedgehog oligonucleotide
primer NS200 F
ctccaggtgctgccgacgctccgggtgcgg
SEQ ID NO:12 - sonic hedgehog oligonucleotide
primer NS146 R
tttatcgattcagctggacttgaccgccatgcccagcgg
SEQ ID NO:13 - sonic hedgehog oligonucleotide
primer NS239
ccctttttctggagactaaataaaatc
SEQ ID NO:14 - GAPDH oligonucleotide primer NS159
ggtcatccctgagctgaacg
SEQ ID NO:15 - GAPDH oligonucleotide primer NS160
ttcgttgtcataccaggaaat

1. A mammalian cell comprising a recombinant sonic hedgehog (SHH) gene such that a recombinant SHH protein can be expressed by the cell, wherein the cell is a stem cell, a fat-derived fibroblast-like cell, a gingival cell, or a periosteum cell, and wherein the SHH protein amino acid sequence is at least 90% homologous to SEQ ID NO:1 or SEQ ID NO:2.

2. The mammalian cell of claim 1, wherein the cell is a stem cell.

3. The mammalian cell of claim 1, wherein the cell is a fat-derived fibroblast-like cell.

4. The mammalian cell of claim 1, wherein the cell is a gingival cell.

5. The mammalian cell of claim 1, wherein the cell is a periosteum cell.

6. The mammalian cell of claim 1, wherein the cell is a human cell.

7-8. (canceled)

9. The mammalian cell of claim 7, wherein the SHH protein is a human protein.

10-11. (canceled)

12. The mammalian cell of claim 1, wherein the cell further comprises a second recombinant gene such that the second recombinant gene can be expressed by the cell, wherein the second recombinant gene encodes a factor that enhances nerve regeneration.

13. (canceled)

14. The mammalian cell of claim 1, wherein the cell further comprises a second recombinant gene such that the second recombinant gene can be expressed by the cell, wherein the second recombinant gene encodes a bone morphogenic protein (BMP).

15-30. (canceled)

31. The mammalian cell of claim 1, wherein the recombinant SHH gene is operably linked to a β-actin enhancer and promoter.

32. The mammalian cell of claim 1, wherein the recombinant SHH gene is operably linked to a β-actin enhancer and promoter, and the mammalian cell is a fat-derived fibroblast-like cell.

33. The mammalian cell of claim 1, wherein the recombinant SHH gene is operably linked to a β-actin enhancer and promoter, and the mammalian cell is a gingival cell.

34. The mammalian cell of claim 1, wherein the recombinant SHH gene is operably linked to a β-actin enhancer and promoter, and the mammalian cell is a periosteum cell.

35-37. (canceled)

38. The mammalian cell of claim 1, wherein the cell is in a matrix suitable for applying to a tissue defect.

39. The mammalian cell of claim 38, wherein the matrix comprises alginate and collagen type I.

40. A matrix suitable for applying to a tissue defect, the matrix comprising alginate and collagen type I.

41-55. (canceled)

56. A tissue regeneration composition comprising the cell of claim 1 in a biocompatible matrix.

57-64. (canceled)

65. A method of regenerating tissue at the site of a tissue defect in a mammal, wherein the tissue is bone, dermis, nervous tissue, or tendon, the method comprising applying the tissue regeneration composition of claim 56 to the defect for a time sufficient to regenerate the tissue.

66-77. (canceled)

78. The method of claim 65, wherein a compound is added to the cell-matrix combination, wherein the compound improves regeneration of the tissue.

79-91. (canceled)

92. A method of regenerating tissue at the site of a tissue defect in a mammal, the method comprising
combining a mammalian cell with a vector,
embedding the cell-vector combination in a matrix suitable for applying to a tissue defect, then
applying the matrix-cell-vector combination to the tissue defect for a time sufficient to regenerate the tissue,
wherein the cell-vector combination is not expanded in culture before the cell-vector-matrix combination is applied to the tissue defect.

93-110. (canceled)


SEQUENCE LISTING (15 sequences)

SEQ ID NO: 1 - protein, 462 aa, Homo sapiens
MLLLARCLLLVLVSSLLVCSGLACGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGGCFPGSATVHLEQGGTKLVKDLSPGDRVLAADDQGRLLYSDFLTFLDRDDGAKKVFYVIETREPRERLLLTAAHLLFVAPHNDSATGEPEASSGSGPPSGGALGPRALFASRVRPGQRVYVVAERDGDRRLLPAAVHSVTLSEEAAGAYAPLTAQGTILINRVLASCYAVIEEHSWAHRAFAPFRLAHALLAALAPARTDRGGDSGGGDRGGGGGRVALTAPGAADAPGAGATAGIHWYSQLLYQIGTWLLDSEALHPLGMAVKSS

SEQ ID NO: 2 - protein, 437 aa, Rattus norvegicus
MLLLLARCFLVALASSLLVCPGLACGPGRGFGKRQHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKARIHCSVKAENSVAAKSDGCFPGSATVHLEQGGTKLVKDLSPGDRVLAADDQGRLLYSDFLTFLDRDEGAKKVFYVIETREPRERLLLTAAHLLFVAPHNDSGPTPGPSPLFASRVRPGQRVYVVAERGGDRRLLPAAVHSVTLREEAAGAYAPLTADGTILINRVLASCYAVIEEHSWAHRAFAPFRLAHALLAALAPARTDGGGGGSIPAPQSVAEARGAGPPAGIHWYSQLLYHIGTWLLDSETLHPLGMAVKSS

SEQ ID NO: 3 - DNA, 39 bp, Artificial
  primer
aaaaagcttgggcgagatgctgctgctggcgagatgtct

SEQ ID NO: 4 - DNA, 24 bp, Artificial
  primer
tcgtcccagccctcggtcacccgc

SEQ ID NO: 5 - DNA, 24 bp, Artificial
  primer
gcgggtgaccgagggctgggacga

SEQ ID NO: 6 - DNA, 18 bp, Artificial
  primer
aggaaagtgaggaagtcg

SEQ ID NO: 7 - DNA, 28 bp, Artificial
  primer
accgcgtgctggcggcggacgaccaggg

SEQ ID NO: 8 - DNA, 34 bp, Artificial
  primer
tgtgcgcgcgggcgccagtgcagccaggagcgcg

SEQ ID NO: 9 - DNA, 91 bp, Artificial
  primer
cccgcgcgcacagatagaggaggagatagtggtggaggtgatagaggaggtggtggaggaagagtagccctaaccgctccaggtgctgccg

SEQ ID NO: 10 - DNA, 91 bp, Artificial
  primer
cggcagcacctggagcggttagggctactcttcctccaccacctcctctatcacctccaccactatctcctcctctatctgtgcgcgcggg

SEQ ID NO: 11 - DNA, 30 bp, Artificial
  primer
ctccaggtgctgccgacgctccgggtgcgg

SEQ ID NO: 12 - DNA, 39 bp, Artificial
  primer
tttatcgattcagctggacttgaccgccatgcccagcgg

SEQ ID NO: 13 - DNA, 27 bp, Artificial
  primer
ccctttttctggagactaaataaaatc

SEQ ID NO: 14 - DNA, 20 bp, Artificial
  primer
ggtcatccctgagctgaacg

SEQ ID NO: 15 - DNA, 21 bp, Artificial
  primer
ttcgttgtcataccaggaaat